SEQUENCE LISTING (148 sequences)

SEQ ID NO: 1 - protein, 11 aa, Artificial Sequence
  Description of Artificial Sequence Synthetic peptide
MTIPCTPTVGY

SEQ ID NO: 2 - protein, 11 aa, Artificial Sequence
  Description of Artificial Sequence Synthetic peptide
SHNISLHPLNN

SEQ ID NO: 3 - protein, 8 aa, Artificial Sequence
  Description of Artificial Sequence Synthetic peptide
VTNKLCLY

SEQ ID NO: 4 - protein, 16 aa, Artificial Sequence
  Description of Artificial Sequence Synthetic peptide
SRNYYFQSFKYLGQGV

SEQ ID NO: 5 - protein, 40 aa, Artificial Sequence
  Description of Artificial Sequence Synthetic polypeptide
KPNNLCYNVSVHFTHQTHCHTTTSSLYPPTSVHDSLEISQ

SEQ ID NO: 6 - protein, 14 aa, Artificial Sequence
  Description of Artificial Sequence Synthetic peptide
THTAVHYAAGNVEA

SEQ ID NO: 7 - protein, 18 aa, Artificial Sequence
  Description of Artificial Sequence Synthetic peptide
AFTSSVSTRTPSLAIAPP

SEQ ID NO: 8 - protein, 14 aa, Artificial Sequence
  Description of Artificial Sequence Synthetic peptide
EEELVPWSRLIITK

SEQ ID NO: 9 - protein, 17 aa, Artificial Sequence
  Description of Artificial Sequence Synthetic peptide
YRQTVYIILTFYIVYRG

SEQ ID NO: 10 - protein, 10 aa, Artificial Sequence
  Description of Artificial Sequence Synthetic peptide
VSDTSVYSTP

SEQ ID NO: 11 - protein, 9 aa, Artificial Sequence
  Description of Artificial Sequence Synthetic peptide
TASTLTALS

SEQ ID NO: 12 - protein, 14 aa, Artificial Sequence
  Description of Artificial Sequence Synthetic peptide
TYSPVTSIAVNCTV

SEQ ID NO: 13 - protein, 7 aa, Artificial Sequence
  Description of Artificial Sequence Synthetic peptide
GTIRVKS

SEQ ID NO: 14 - protein, 8 aa, Artificial Sequence
  Description of Artificial Sequence Synthetic peptide
NCPNVVWY

SEQ ID NO: 15 - protein, 8 aa, Artificial Sequence
  Description of Artificial Sequence Synthetic peptide
THGHHIYP

SEQ ID NO: 16 - protein, 32 aa, Artificial Sequence
  Description of Artificial Sequence Synthetic polypeptide
QTPTYQHKILTSHPICHPDVSSPAAYHDLCRS

SEQ ID NO: 17 - protein, 7 aa, Artificial Sequence
  Description of Artificial Sequence Synthetic peptide
YSRRCYK

SEQ ID NO: 18 - protein, 10 aa, Artificial Sequence
  Description of Artificial Sequence Synthetic peptide
TTPLCPRYVG

SEQ ID NO: 19 - protein, 12 aa, Artificial Sequence
  Description of Artificial Sequence Synthetic peptide
NVSSAVTTTVQT

SEQ ID NO: 20 - protein, 7 aa, Artificial Sequence
  Description of Artificial Sequence Synthetic peptide
ASTSVIA

SEQ ID NO: 21 - protein, 29 aa, Artificial Sequence
  Description of Artificial Sequence Synthetic peptide
EGHLYTVNCEASYSYDQVSLNATCKVILL

SEQ ID NO: 22 - protein, 11 aa, Artificial Sequence
  Description of Artificial Sequence Synthetic peptide
PDILSVTCYAR

SEQ ID NO: 23 - protein, 13 aa, Artificial Sequence
  Description of Artificial Sequence Synthetic peptide
CKGPFTQVGYLSA

SEQ ID NO: 24 - protein, 8 aa, Artificial Sequence
  Description of Artificial Sequence Synthetic peptide
GKLHLSYN

SEQ ID NO: 25 - protein, 8 aa, Artificial Sequence
  Description of Artificial Sequence Synthetic peptide
AQELLISG

SEQ ID NO: 26 - protein, 7 aa, Artificial Sequence
  Description of Artificial Sequence Synthetic peptide
TEYTCSF

SEQ ID NO: 27 - protein, 12 aa, Artificial Sequence
  Description of Artificial Sequence Synthetic peptide
DLFTYPIYAVYG

SEQ ID NO: 28 - protein, 38 aa, Artificial Sequence
  Description of Artificial Sequence Synthetic polypeptide
MRVRVLLQEHEHCLLNGSSLYHPNSTVHLHQGDQLIPP

SEQ ID NO: 29 - protein, 22 aa, Artificial Sequence
  Description of Artificial Sequence Synthetic peptide
LREFVFYLNGTYTVVRLHVQIA

SEQ ID NO: 30 - protein, 10 aa, Artificial Sequence
  Description of Artificial Sequence Synthetic peptide
TTTYVFIKSD

SEQ ID NO: 31 - protein, 15 aa, Artificial Sequence
  Description of Artificial Sequence Synthetic peptide
SNSTCRLNVTELASI

SEQ ID NO: 32 - protein, 12 aa, Artificial Sequence
  Description of Artificial Sequence Synthetic peptide
LHGMCISICYYE

SEQ ID NO: 33 - protein, 7 aa, Artificial Sequence
  Description of Artificial Sequence Synthetic peptide
EIIGVAF

SEQ ID NO: 34 - protein, 10 aa, Artificial Sequence
  Description of Artificial Sequence Synthetic peptide
HNESVVDLWL

SEQ ID NO: 35 - protein, 10 aa, Artificial Sequence
  Description of Artificial Sequence Synthetic peptide
KMRTVPVTKL

SEQ ID NO: 36 - protein, 9 aa, Artificial Sequence
  Description of Artificial Sequence Synthetic peptide
TVGRYDCLR

SEQ ID NO: 37 - protein, 15 aa, Artificial Sequence
  Description of Artificial Sequence Synthetic peptide
IIERLYVRLGSLYPR

SEQ ID NO: 38 - protein, 13 aa, Artificial Sequence
  Description of Artificial Sequence Synthetic peptide
PGSGLAKHPSVSA

SEQ ID NO: 39 - protein, 29 aa, Artificial Sequence
  Description of Artificial Sequence Synthetic peptide
TTAGVTSAHGPLCPLVFQGWAYAVYHQGD

SEQ ID NO: 40 - protein, 12 aa, Artificial Sequence
  Description of Artificial Sequence Synthetic peptide
VLMTLDVYCCRQ

SEQ ID NO: 41 - protein, 10 aa, Artificial Sequence
  Description of Artificial Sequence Synthetic peptide
SSNTVVAFSH

SEQ ID NO: 42 - protein, 8 aa, Artificial Sequence
  Description of Artificial Sequence Synthetic peptide
ADNTLLIE

SEQ ID NO: 43 - protein, 27 aa, Artificial Sequence
  Description of Artificial Sequence Synthetic peptide
HVDGISCQDHFRAQHQDCPAQTVHVRG

SEQ ID NO: 44 - protein, 32 aa, Artificial Sequence
  Description of Artificial Sequence Synthetic polypeptide
AFGLTHLQSCCLNEHSQLSERVAYHLKLRPAT

SEQ ID NO: 45 - protein, 33 aa, Artificial Sequence
  Description of Artificial Sequence Synthetic polypeptide
AMYTVGILALGSFSSFYSQIARSLGVLPNDHHY

SEQ ID NO: 46 - protein, 16 aa, Artificial Sequence
  Description of Artificial Sequence Synthetic peptide
PKTLLSLSPRQACVPR

SEQ ID NO: 47 - protein, 7 aa, Artificial Sequence
  Description of Artificial Sequence Synthetic peptide
SHRPVCY

SEQ ID NO: 48 - protein, 8 aa, Artificial Sequence
  Description of Artificial Sequence Synthetic peptide
FAHQCLQA

SEQ ID NO: 49 - protein, 35 aa, Artificial Sequence
  Description of Artificial Sequence Synthetic polypeptide
GRLTCQRVRRLLPCDLDIHPSHRLLTLMNNCVCDG

SEQ ID NO: 50 - protein, 7 aa, Artificial Sequence
  Description of Artificial Sequence Synthetic peptide
VWNAFRL

SEQ ID NO: 51 - protein, 11 aa, Artificial Sequence
  Description of Artificial Sequence Synthetic peptide
KKPLKLANYRA

SEQ ID NO: 52 - protein, 7 aa, Artificial Sequence
  Description of Artificial Sequence Synthetic peptide
TRTLVTR

SEQ ID NO: 53 - protein, 16 aa, Artificial Sequence
  Description of Artificial Sequence Synthetic peptide
NTSHHSVVWQRYDIYS

SEQ ID NO: 54 - protein, 8 aa, Artificial Sequence
  Description of Artificial Sequence Synthetic peptide
MPPLCIIT

SEQ ID NO: 55 - protein, 19 aa, Artificial Sequence
  Description of Artificial Sequence Synthetic peptide
NLTLYNLTVKDTGVYLLQD

SEQ ID NO: 56 - protein, 20 aa, Artificial Sequence
  Description of Artificial Sequence Synthetic peptide
YTGDVEAFYLIIHPRSFCRA

SEQ ID NO: 57 - protein, 17 aa, Artificial Sequence
  Description of Artificial Sequence Synthetic peptide
ETRRCFYPGPGRVVVTD

SEQ ID NO: 58 - protein, 10 aa, Artificial Sequence
  Description of Artificial Sequence Synthetic peptide
SSSRICPLSN

SEQ ID NO: 59 - protein, 8 aa, Artificial Sequence
  Description of Artificial Sequence Synthetic peptide
KSVRLPQY

SEQ ID NO: 60 - protein, 28 aa, Artificial Sequence
  Description of Artificial Sequence Synthetic peptide
DVSGYRVSSSVSECYVQHGVLVAAWLVR

SEQ ID NO: 61 - protein, 7 aa, Artificial Sequence
  Description of Artificial Sequence Synthetic peptide
THFKVGA

SEQ ID NO: 62 - protein, 45 aa, Artificial Sequence
  Description of Artificial Sequence Synthetic polypeptide
TELPQVDARLSYVMLTVYPCSACNRSVLHCRPASRLPWLPLRVTP

SEQ ID NO: 63 - protein, 10 aa, Artificial Sequence
  Description of Artificial Sequence Synthetic peptide
VLRGVLQPAS

SEQ ID NO: 64 - protein, 10 aa, Artificial Sequence
  Description of Artificial Sequence Synthetic peptide
IMDYVELATR

SEQ ID NO: 65 - protein, 16 aa, Artificial Sequence
  Description of Artificial Sequence Synthetic peptide
LTMRLGILPLFIIAFF

SEQ ID NO: 66 - protein, 70 aa, Artificial Sequence
  Description of Artificial Sequence Synthetic polypeptide
DSFDYLVERCQQSCHGHFVRRLVQALKRAMYSVELAVCYFSTSVRDVAEAVKKSSSRCYADATSAAVVVT

SEQ ID NO: 67 - protein, 16 aa, Artificial Sequence
  Description of Artificial Sequence Synthetic peptide
PGTTIDVSAESSSVLC

SEQ ID NO: 68 - protein, 17 aa, Artificial Sequence
  Description of Artificial Sequence Synthetic peptide
MLHDLFCGCHYPEKCRR

SEQ ID NO: 69 - protein, 7 aa, Artificial Sequence
  Description of Artificial Sequence Synthetic peptide
YGSGCRF

SEQ ID NO: 70 - protein, 7 aa, Artificial Sequence
  Description of Artificial Sequence Synthetic peptide
PAPPALS

SEQ ID NO: 71 - protein, 18 aa, Artificial Sequence
  Description of Artificial Sequence Synthetic peptide
DAVHVAVQAAVQATVQVS

SEQ ID NO: 72 - protein, 15 aa, Artificial Sequence
  Description of Artificial Sequence Synthetic peptide
MFSYLAKLGTYHHYR

SEQ ID NO: 73 - protein, 9 aa, Artificial Sequence
  Description of Artificial Sequence Synthetic peptide
NGTLSVILN

SEQ ID NO: 74 - protein, 12 aa, Artificial Sequence
  Description of Artificial Sequence Synthetic peptide
APPVVRSPCLQP

SEQ ID NO: 75 - protein, 8 aa, Artificial Sequence
  Description of Artificial Sequence Synthetic peptide
GSPQLLPY

SEQ ID NO: 76 - protein, 11 aa, Artificial Sequence
  Description of Artificial Sequence Synthetic peptide
DRLEVACIFPA

SEQ ID NO: 77 - protein, 39 aa, Artificial Sequence
  Description of Artificial Sequence Synthetic polypeptide
DWPEVSIRVHLCYWPEIVRSLVVDARSGQVLHNDASCYI

SEQ ID NO: 78 - protein, 13 aa, Artificial Sequence
  Description of Artificial Sequence Synthetic peptide
AAQRLSLSFRLIT

SEQ ID NO: 79 - protein, 8 aa, Artificial Sequence
  Description of Artificial Sequence Synthetic peptide
GTYTCVLG

SEQ ID NO: 80 - protein, 11 aa, Artificial Sequence
  Description of Artificial Sequence Synthetic peptide
TTALVADVHDL

SEQ ID NO: 81 - protein, 9 aa, Artificial Sequence
  Description of Artificial Sequence Synthetic peptide
SDRSCDLAF

SEQ ID NO: 82 - protein, 7 aa, Artificial Sequence
  Description of Artificial Sequence Synthetic peptide
QTRYLWT

SEQ ID NO: 83 - protein, 17 aa, Artificial Sequence
  Description of Artificial Sequence Synthetic peptide
RHRVVHYIPGTSGLLPS

SEQ ID NO: 84 - protein, 10 aa, Artificial Sequence
  Description of Artificial Sequence Synthetic peptide
RELCVPFISQ

SEQ ID NO: 85 - protein, 7 aa, Artificial Sequence
  Description of Artificial Sequence Synthetic peptide
RRYHLRR

SEQ ID NO: 86 - protein, 10 aa, Artificial Sequence
  Description of Artificial Sequence Synthetic peptide
MIRGVLEVHT

SEQ ID NO: 87 - protein, 9 aa, Artificial Sequence
  Description of Artificial Sequence Synthetic peptide
IMEPQVLDF

SEQ ID NO: 88 - protein, 9 aa, Artificial Sequence
  Description of Artificial Sequence Synthetic peptide
TEHGLLVSM

SEQ ID NO: 89 - protein, 22 aa, Artificial Sequence
  Description of Artificial Sequence Synthetic peptide
YRSELLCTSAFLGYSAVFLLET

SEQ ID NO: 90 - protein, 38 aa, Artificial Sequence
  Description of Artificial Sequence Synthetic polypeptide
AVTQVRLSDLRLKHRCGIVKADNLLHFALCTVISCVEN

SEQ ID NO: 91 - protein, 18 aa, Artificial Sequence
  Description of Artificial Sequence Synthetic peptide
LTRKCLHDLLQYLDAVNV

SEQ ID NO: 92 - protein, 21 aa, Artificial Sequence
  Description of Artificial Sequence Synthetic peptide
FGRLLHHSARRLICSALYLLF

SEQ ID NO: 93 - protein, 16 aa, Artificial Sequence
  Description of Artificial Sequence Synthetic peptide
EPHIVQYVPATFVLFQ

SEQ ID NO: 94 - protein, 15 aa, Artificial Sequence
  Description of Artificial Sequence Synthetic peptide
TRHTCLQLVARFFFR

SEQ ID NO: 95 - protein, 8 aa, Artificial Sequence
  Description of Artificial Sequence Synthetic peptide
EAHSFSLK

SEQ ID NO: 96 - protein, 14 aa, Artificial Sequence
  Description of Artificial Sequence Synthetic peptide
DGWPVGLGLLDVLN

SEQ ID NO: 97 - protein, 10 aa, Artificial Sequence
  Description of Artificial Sequence Synthetic peptide
YPNLPSPPKL

SEQ ID NO: 98 - protein, 10 aa, Artificial Sequence
  Description of Artificial Sequence Synthetic peptide
YPNLPSPPKL

SEQ ID NO: 99 - protein, 14 aa, Artificial Sequence
  Description of Artificial Sequence Synthetic peptide
EPNYVAPPARQFRF

SEQ ID NO: 100 - protein, 27 aa, Artificial Sequence
  Description of Artificial Sequence Synthetic peptide
PLNNVSSYQASCVVKDGVLDAVWRVQG

SEQ ID NO: 101 - protein, 8 aa, Artificial Sequence
  Description of Artificial Sequence Synthetic peptide
PEKGIVAR

SEQ ID NO: 102 - protein, 38 aa, Artificial Sequence
  Description of Artificial Sequence Synthetic polypeptide
RLHAPECLVETTEAVFRLRQWVPTDLDHLTLHLVPCTK

SEQ ID NO: 103 - protein, 13 aa, Artificial Sequence
  Description of Artificial Sequence Synthetic peptide
KPMWCQPRYHIRY

SEQ ID NO: 104 - protein, 12 aa, Artificial Sequence
  Description of Artificial Sequence Synthetic peptide
QGATYQLSIVRQ

SEQ ID NO: 105 - protein, 9 aa, Artificial Sequence
  Description of Artificial Sequence Synthetic peptide
AGFQVRAAS

SEQ ID NO: 106 - protein, 42 aa, Artificial Sequence
  Description of Artificial Sequence Synthetic polypeptide
NAVDLDRPPLWSGSLPHLPVYDVRSPRPLRPPSSQHHAVSPE

SEQ ID NO: 107 - protein, 10 aa, Artificial Sequence
  Description of Artificial Sequence Synthetic peptide
QYQELQYLVE

SEQ ID NO: 108 - protein, 13 aa, Artificial Sequence
  Description of Artificial Sequence Synthetic peptide
IPRPSFPPPDPPS

SEQ ID NO: 109 - protein, 7 aa, Artificial Sequence
  Description of Artificial Sequence Synthetic peptide
AESTVSH

SEQ ID NO: 110 - protein, 9 aa, Artificial Sequence
  Description of Artificial Sequence Synthetic peptide
SRDSLLLTR

SEQ ID NO: 111 - protein, 29 aa, Artificial Sequence
  Description of Artificial Sequence Synthetic peptide
GLRQLRQQLTVRWQLFRLRCHGWTQQVSS

SEQ ID NO: 112 - protein, 9 aa, Artificial Sequence
  Description of Artificial Sequence Synthetic peptide
ESNVVSQTA

SEQ ID NO: 113 - protein, 7 aa, Artificial Sequence
  Description of Artificial Sequence Synthetic peptide
RTWFVQR

SEQ ID NO: 114 - protein, 15 aa, Artificial Sequence
  Description of Artificial Sequence Synthetic peptide
EAQELAIIPPAPTVL

SEQ ID NO: 115 - protein, 9 aa, Artificial Sequence
  Description of Artificial Sequence Synthetic peptide
EVQEPQVTY

SEQ ID NO: 116 - protein, 10 aa, Artificial Sequence
  Description of Artificial Sequence Synthetic peptide
NTLTVACPPR

SEQ ID NO: 117 - protein, 20 aa, Artificial Sequence
  Description of Artificial Sequence Synthetic peptide
PHRALFRLCLGLWVSSYLVR

SEQ ID NO: 118 - protein, 14 aa, Artificial Sequence
  Description of Artificial Sequence Synthetic peptide
SGVGSSPPSSCVPM

SEQ ID NO: 119 - protein, 8 aa, Artificial Sequence
  Description of Artificial Sequence Synthetic peptide
PGHGVHRV

SEQ ID NO: 120 - protein, 13 aa, Artificial Sequence
  Description of Artificial Sequence Synthetic peptide
PERLLLSQIPVER

SEQ ID NO: 121 - protein, 7 aa, Artificial Sequence
  Description of Artificial Sequence Synthetic peptide
ALTELEY

SEQ ID NO: 122 - protein, 7 aa, Artificial Sequence
  Description of Artificial Sequence Synthetic peptide
VWRAAFL

SEQ ID NO: 123 - protein, 19 aa, Artificial Sequence
  Description of Artificial Sequence Synthetic peptide
AGTLLPLGRPYGFYARVTP

SEQ ID NO: 124 - protein, 16 aa, Artificial Sequence
  Description of Artificial Sequence Synthetic peptide
DAWIVLVATVVHEVDP

SEQ ID NO: 125 - protein, 25 aa, Artificial Sequence
  Description of Artificial Sequence Synthetic peptide
HPEGLCAQDGLYLALGAGFRVFVYD

SEQ ID NO: 126 - protein, 8 aa, Artificial Sequence
  Description of Artificial Sequence Synthetic peptide
NNTLILAA

SEQ ID NO: 127 - protein, 13 aa, Artificial Sequence
  Description of Artificial Sequence Synthetic peptide
GAGEVVRLYRCNR

SEQ ID NO: 128 - protein, 16 aa, Artificial Sequence
  Description of Artificial Sequence Synthetic peptide
RATLLPQPALRQTLLR

SEQ ID NO: 129 - protein, 7 aa, Artificial Sequence
  Description of Artificial Sequence Synthetic peptide
GTTVALQ

SEQ ID NO: 130 - protein, 34 aa, Artificial Sequence
  Description of Artificial Sequence Synthetic polypeptide
LQPMVLLGAWQELAQYEPFASAPHPASLLTAVRR

SEQ ID NO: 131 - protein, 25 aa, Artificial Sequence
  Description of Artificial Sequence Synthetic peptide
LNQRLCCGWLALGAVLPARWLGCAA

SEQ ID NO: 132 - protein, 21 aa, Artificial Sequence
  Description of Artificial Sequence Synthetic peptide
GDAPCAMAGAVGSAVTIPPQP

SEQ ID NO: 133 - protein, 17 aa, Artificial Sequence
  Description of Artificial Sequence Synthetic peptide
GSAICVPNADAHAVVGA

SEQ ID NO: 134 - protein, 16 aa, Artificial Sequence
  Description of Artificial Sequence Synthetic peptide
ATAAAAAAAAAPTVMV

SEQ ID NO: 135 - protein, 46 aa, Artificial Sequence
  Description of Artificial Sequence Synthetic polypeptide
PRAMLVVVLDELGAVFGYCPLDGHVYPLAAELSHFLRAGVLGALAL

SEQ ID NO: 136 - protein, 8 aa, Artificial Sequence
  Description of Artificial Sequence Synthetic peptide
AARRLLPE

SEQ ID NO: 137 - protein, 14 aa, Artificial Sequence
  Description of Artificial Sequence Synthetic peptide
WDALHLHPRAALWA

SEQ ID NO: 138 - protein, 9 aa, Artificial Sequence
  Description of Artificial Sequence Synthetic peptide
IHDPVAFRL

SEQ ID NO: 139 - protein, 9 aa, Artificial Sequence
  Description of Artificial Sequence Synthetic peptide
RTLGLDLTT

SEQ ID NO: 140 - protein, 14 aa, Artificial Sequence
  Description of Artificial Sequence Synthetic peptide
QSQLPEKYIGFYQI

SEQ ID NO: 141 - protein, 16 aa, Artificial Sequence
  Description of Artificial Sequence Synthetic peptide
TMPPPLSAQASVSYAL

SEQ ID NO: 142 - protein, 8 aa, Artificial Sequence
  Description of Artificial Sequence Synthetic peptide
RPLSTVDD

SEQ ID NO: 143 - protein, 7 aa, Artificial Sequence
  Description of Artificial Sequence Synthetic peptide
ESHWVLG

SEQ ID NO: 144 - protein, 12 aa, Artificial Sequence
  Description of Artificial Sequence Synthetic peptide
RPMPVVPEECYD

SEQ ID NO: 145 - protein, 11 aa, Artificial Sequence
  Description of Artificial Sequence Synthetic peptide
EGHQVIPLCAS

SEQ ID NO: 146 - protein, 8 aa, Artificial Sequence
  Description of Artificial Sequence Synthetic peptide
KPPRLCKT

SEQ ID NO: 147 - protein, 7 aa, Artificial Sequence
  Description of Artificial Sequence Synthetic peptide
GPPPLPP

SEQ ID NO: 148 - protein, 10 aa, Artificial Sequence
  Description of Artificial Sequence Synthetic peptide
RPKKCQTHAP

(19) United States
(12) Patent Application Publication
(10) Pub. No.: US 2016/0289303 A1
(43) Pub. Date: Oct. 6, 2016

(54) METHODS AND COMPOSITIONS FOR THE TREATMENT OF HCMV

(71) Applicants: PRESIDENT AND FELLOWS OF HARVARD COLLEGE, Boston, MA (US); CAMBRIDGE UNIVERSITY, Cambridge (GB); CARDIFF UNIVERSITY, Cardiff (GB)

(72) Inventors: MICHAEL P. WEEKES, Boston, MA (US); STEVEN P. GYGI, Foxborough, MA (US); PAUL J. LEHNER, Cambridge (GB); GAVIN W. WILKINSON, Cardiff (GB); PETER TOMASEC, Cardiff (GB); RICHARD J. STANTON, Cardiff (GB)

(21) Appl. No.: 15/036,092

(22) PCT Filed: Nov. 14, 2014
(86) PCT No.: PCT/US14/65645

§ 371 Date: May 12, 2016

Related U.S. Application Data
(60) Provisional application No. 61/904,646, filed on Nov. 15, 2013.

(51) Int. Cl. C07K 16/08; A61K 47/48; A61K 31/7048

(52) U.S. Cl. CPC C07K 16/088; A61K 31/7048; A61K 47/48523; C07K 2317/92; C07K 2317/732; C07K 2317/34

(57) ABSTRACT

Provided herein are compositions and methods for the treatment of HCMV infection in a subject.

### RELATED APPLICATIONS

This application claims the benefit of priority to U.S. Provisional Patent Application Ser. No. 61/904,646, filed Nov. 15, 2013, which is hereby incorporated by reference in its entirety.

### GOVERNMENT INTEREST

This invention was made with Government support under National Institutes of Health Grants GM067945 and HG006673. The Government has certain rights in the invention.

### BACKGROUND

Human Cytomegalovirus (HCMV, also known as human herpesvirus-5) is a nearly ubiquitous herpes virus that infects between 60% and 90% of individuals. Following primary infection, HCMV typically establishes a persistent infection that is kept under control by a healthy immune system. HCMV employs a multitude of immune-modulatory strategies to evade the host immune response. Examples of such strategies include inhibition of interferon (IFN) and IFN-stimulated genes, degradation of HLA to prevent antigen presentation to cytotoxic T cells and modulation of activating and inhibitory ligands to prevent natural killer (NK) cell function.

Though HCMV infection typically goes unnoticed in healthy individuals, reactivation from viral latency in immunocompromised individuals (e.g., HIV-infected persons, organ transplant recipients), or acquisition of primary infection in such individuals (e.g., during transplantation) can lead to serious disease. For example, HCMV is one of the major causes of graft failure and mortality in transplant recipients who require prolonged immunosuppression, and HCMV infection during pregnancy can lead to congenital abnormalities. HCMV infection has also been linked with mucoepidermoid carcinoma, even in immunocompetent individuals.

HCMV infection in immunocompromised individuals is currently treated using purified plasma immunoglobulin (CMV-IGIV) and antiviral drugs, such as Ganciclovir (Cytovene) and Valganciclovir (Valcyte). Because CMV-IVIG is derived from donated human plasma, it is difficult to produce in large quantity and its use carries the risk of the transmission of infectious disease. Drug-resistant HCMV strains have become increasingly common, often rendering current therapies ineffective. Recent attempts to develop an HCMV vaccine have proven unsuccessful. Thus, there is a great need for new and improved methods and compositions for the treatment of HCMV.

### SUMMARY

Provided herein are compositions and methods for the treatment of HCMV infection in a subject.

In certain aspects, provided herein are methods of treating HCMV infection that include the step of administering to a subject an agent that specifically binds to a target protein expressed on the plasma membrane of HCMV infected cells. In some embodiments, the target protein is an HCMV protein, such as the proteins encoded by the genes listed in Table 1 and/or Table 2. In some embodiments, the target protein is an endogenous protein that has upregulated plasma membrane expression following HCMV infection, such as the proteins encoded by the genes listed in Table 3 and/or Table 4. In some embodiments, the agent binds to an epitope listed in Table 5.

In some embodiments of the methods provided herein, the agent is an antibody (e.g., a full-length antibody or an antigen binding fragment thereof). In some embodiments, the antibody is a monoclonal antibody or a polyclonal antibody. In some embodiments, the antibody is a chimeric antibody, a humanized antibody or a fully human antibody. In some embodiments, the antibody is a full length immunoglobulin molecule, an scFv, a Fab fragment, an Fab′ fragment, a F(ab′)2 fragment, an Fv, a NANOBODY® or a disulfide linked Fv. In some embodiments, the antibody binds to the target protein with a dissociation constant of no greater than about 10⁻⁷ M, 10⁻⁸ M or 10⁻⁹M. In some embodiments, the antibody binds to an extracellular epitope of the target protein. In some embodiments, the antibody binds to an epitope listed in Table 5.

In some embodiments of the methods provided herein, the antibody is part of an antibody-drug conjugate. In some embodiments, the antibody is linked to a cytotoxic agent (e.g., MMAE, DM-1, a maytansinoid, a doxorubicin derivative, a auristatin, a calcheamicin, CC-1065, aduocarmycin or a anthracycline). In some embodiments, the antibody is linked to an antiviral agent (e.g., ganciclovir, valganciclovir, foscarnet, cidofovir, acyclovir, formivirsen, maribavir, BAY 38-4766 or GW275175X).

In certain aspects, provided herein are antibodies that specifically bind to an extracellular epitope of a protein expressed on the plasma membrane of HCMV infected cells (e.g., an epitope listed in Table 5). In some embodiments, the target protein is an HCMV protein, such as the proteins encoded by the genes listed in Table 1 and/or Table 2. In some embodiments, the target protein is an endogenous protein that has upregulated plasma membrane expression following HCMV infection, such as the proteins encoded by the genes listed in Table 3 and/or Table 4

In some embodiments of the antibodies provided herein, the antibody is a monoclonal antibody or a polyclonal antibody. In some embodiments, the antibody is a chimeric antibody, a humanized antibody or a fully human antibody. In some embodiments, the antibody is a full length immunoglobulin molecule, an scFv, a Fab fragment, an Fab′ fragment, a F(ab′)2 fragment, an Fv, a NANOBODY® or a disulfide linked Fv. In some embodiments, the antibody binds to the target protein with a dissociation constant of no greater than about 10⁻⁷ M, 10⁻⁸ M or 10⁻⁹M. In some embodiments, the antibody binds to an extracellular epitope of the target protein. In some embodiments, the epitope is an epitope listed in Table 5.

In some embodiments of the antibodies provided herein, the antibody is part of an antibody-drug conjugate. In some embodiments, the antibody is linked to a cytotoxic agent (e.g., MMAE, DM-1, a maytansinoid, a doxorubicin derivative, an auristatin, a calcheamicin, CC-1065, an aduocarmycin or an anthracycline). In some embodiments, the antibody is linked to an antiviral agent (e.g., ganciclovir, valganciclovir, foscarnet, cidofovir, acyclovir, formivirsen, maribavir, BAY 38-4766 or GW275175X).

In certain aspects, provided herein are methods of treating HCMV infection that include the step of administering to a subject a cytotoxic agent to which a transport protein provides cellular resistance, wherein plasma membrane expression of the transport protein is downregulated following HCMV infection. In some embodiments, the transport protein is encoded by ABCC3, SLC38A4 or SLC2A10. In some embodiments the agent is Etoposide.

### BRIEF DESCRIPTION OF THE DRAWINGS

FIG. 1 is a schematic showing the workflow of experiments PM1, PM2, WCL1 and WCL2 of the Exemplification. PM1 and PM2 refer to independent experiments in which quantitative temporal viromics were used to examine protein expression at the plasma membrane of HCMV infected cells. WCL1 and WCL2 refer to independent experiments in which the protein expression in whole cell lysates of HCMV infected cells was examined.

FIG. 2 shows the relative abundance of ABC transporters in mock infected cells and in infected cells at 24, 48 and 72 hours after HCMV infection.

FIG. 3 shows the relative abundance of HCMV proteins in mock infected cells and in infected cells at 24, 48 and 72 hours after HCMV infection. gB, gO, gH and gL are virion glycoproteins expressed late in infection.

FIG. 4 shows a principal component analysis of quantified proteins from experiments PM1 and WCL1.

FIG. 5 is a table listing endogenous proteins that have upregulated plasma membrane expression following HCMV infection.

FIG. 6 shows the temporal modulation of cell surface immunoreceptors. 6A and 6B show temporal profiles of NK ligands (A) or T-cell ligands (B). C shows temporal profiles of γ-protocadherins.

FIG. 7 is a table listing proteins quantified in either experiment PM1 or PM2 that have an Interpro annotation of butyrophylin, c-type lectin, immunoglobulin, Ig, MHC or TNF and that exhibit a greater than 4-fold modulation in plasma membrane expression following HCMV infection.

FIG. 8 is a table listing functional protein categories that were enriched among the proteins that were highly downregulated at the plasma membrane following HCMV infection.

FIG. 9 shows temporal classes of HCMV gene expression. In 9A, the k-means method was used to cluster all quantified HCMV proteins into 4 or 5 classes. Shown are the average temporal profiles of each class. With 4 classes, proteins grouped into the classical cascade of a, b, g1, g2 gene expression. With 5 classes, a distinct temporal profile appeared, with maximal expression at 48 h but little expression before or after this time. 9B depicts the number of temporal classes of HCMV gene expression. The summed distance of each protein from its cluster centroid was calculated for 1-14 classes and plotted. The point of inflexion fell between 5-7 classes. In 9C, temporal profiles of proteins in each k-means class were subjected to hierarchical clustering by Euclidian distance. 9D depicts temporal profiles of the central protein of each cluster (upper panels), and all new ORFs quantified by QTV (lower panels).

FIG. 10 shows the changes in plasma membrane expression of canonical HCMV proteins following HCMV infection.

FIG. 11 is a table listing the origin of g1b proteins quantified. “Genetic Region” refers to the region of the viral genome from which the specified gene originates, listed in kb. The listed “Start” and “Stop” positions are with reference to the Merlin strain HCMV genome nucleic acid sequence provided at NCBI Reference number NC 006273.2.

FIG. 12 shows the relationship between four novel ORFs and the associated canonical HCMV counterparts, with temporal profiles.

FIG. 13 is a table listing 9 new ORFs quantified. It was not possible to distinguish between ORFL184C.iORF3 and ORFL185C, or between ORFL294W.iORF1 and ORFL294W on the basis of the identified peptides. The listed “Start” and “Stop” positions are with reference to the Merlin strain HCMV genome nucleic acid sequence provided at NCBI Reference number NC 006273.2.

FIG. 14 is a table listing 67 HCMV proteins detected at the cell surface in experiments PM1 or PM2. A peptide ratio cutoff for ‘high confidence’ PM viral proteins was determined (bold line between UL141 and UL14). The temporal class of protein expression is shown.

FIG. 15 shows data related to the HCMV proteins quantified at the surface of infected fibroblasts. 15A is a histogram of peptide ratios for all GO-annotated proteins quantified in experiments PM1 or PM2. The proteins indicated as “PM Only” were not detected in experiments WCL1 or WCL2. 15B depicts temporal profiles of all ‘high confidence’ PM proteins. Virion envelope glycoproteins were generally detected significantly earlier in whole cell lysates than in plasma membrane samples.

FIG. 16 shows temporal profiles of ‘high confidence’ PM proteins detected in experiment PM1. Known virion envelope glycoproteins (starred) were generally detected significantly earlier in whole cell lysates than in plasma membrane samples. Values shown are averages of two biological replicates, +/− range.

FIG. 17 shows temporal profiles and normalized abundance of selected PM proteins. The top panels depict the relative abundance of the selected PM proteins as determined in an 8-plex TMT experiment in biological duplicate at 4 time points of HCMV infection. The middle panels depict the relative abundance of the selected PM proteins as determined in a 10-plex TMT, 8-time-point analysis. The bottom panel depicts the normalized spectral abundance of the selected PM proteins, as well as the relative abundance of known cell surface/virion glycoproteins gM, gB and gN.

FIG. 18 shows that serum from HCMV seropositive individuals induces antibody-dependent cellular cytotoxicity. Fibroblasts were infected with HCMV strain Merlin. After 48 or 72 hours, serum from HCMV seropositive (sero+) or seronegative (sero−) donors was added to the culture along with NK cells, and the level of NK degranulation assessed via a CD107a assay.

### DETAILED DESCRIPTION

### General

Disclosed herein are novel compositions and methods for the treatment of HCMV infection.

As described herein, a new proteomic approach was used to study temporal changes in plasma membrane expression of viral and endogenous proteins following HCMV infection. Accurate multiplexed quantitative measurement of protein abundance using triple-stage mass spectrometry (MS3) to measure ten isobaric chemical reporters (tandem mass tags, TMT). The TMT-based process was combined with plasma membrane profiling (PMP), a method for isolation of highly purified plasma membrane proteins for proteomic analysis. In total, 1,184 cell surface receptors were quantified over eight time points during productive infection of primary human fibroblasts with HCMV. Through simultaneous analysis of lysates of infected cells, expression of 7,491 host proteins and 80% of all canonical viral proteins was quantified, providing a near-complete view of the host proteome and HCMV virome over time following HCMV infection.

Using the above approach, proteins for which plasma membrane expression was rapidly upregulated following HCMV expression were identified (e.g., the proteins encoded by the genes listed in Tables 1-4). Therapeutic agents that selectively bind to such proteins (e.g., therapeutic antibodies) can be used to selectively target virus infected cells for the treatment of HCMV infection.

As described herein, HCMV infection induces the downregulation of the plasma membrane expression of numerous endogenous proteins, including many involved in the host immune response (including natural killer cell ligands and T-cell costimulatory molecules). HCMV proteins present on the plasma membrane (e.g., the proteins encoded by the genes listed in Tables 1 and 2) may facilitate this process by binding to and internalizing the endogenous proteins (e.g., via the endosome network). Indeed, a vast majority of the plasma membrane expressed HCMV proteins disclosed herein contain amino acid sequences that correspond to sorting signals known to facilitate protein movement through the endosome network. Internalization of an agent (e.g., an anti-viral or a cytotoxic agent) by an HCMV infected cell can therefore be facilitated by linking the agent to an antibody that binds to an extracellular epitope of a plasma membrane expressed HCMV protein (e.g., a protein encoded by a gene listed in Tables 1 and 2), which would then shuttle the antibody and agent into the cell as it would its endogenous protein target.

Thus, in certain embodiments, provided herein are methods and compositions for treating HCMV infection by targeting a protein selectively expressed on the plasma membrane of HCMV infected cells (e.g., the proteins encoded by the genes listed in Tables 1-4). In some embodiments, provided herein are antibodies that specifically bind to an extracellular epitope of a protein selectively expressed on the plasma membrane of HCMV infected cells (e.g., an extracellular epitope of proteins encoded by the genes listed in Tables 1-4, such as the epitopes listed in Table 5). In some embodiments, provided here are methods of treating HCMV infection by administering a cytotoxic agent for which cellular resistance is conveyed by a protein that is rapidly downregulated on the plasma membrane of HCMV infected cells.

### DEFINITIONS

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, an antibody or antigen binding fragment thereof described herein.

The term “agent” is used herein to denote a chemical compound, a small molecule, a mixture of chemical compounds and/or a biological macromolecule (such as a nucleic acid, an antibody, an antibody fragment, a protein or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

The term “binding” or “interacting” refers to an association, which may be a stable association, between two molecules, e.g., between a polypeptide and a binding partner or agent, e.g., small molecule, due to, for example, electrostatic, hydrophobic, ionic and/or hydrogen-bond interactions under physiological conditions.

The terms “CDR”, and its plural “CDRs”, refer to a complementarity determining region (CDR) of an antibody or antibody fragment, which determine the binding character of an antibody or antibody fragment. In most instances, three CDRs are present in a light chain variable region (CDRL1, CDRL2 and CDRL3) and three CDRs are present in a heavy chain variable region (CDRH1, CDRH2 and CDRH3). CDRs contribute to the functional activity of an antibody molecule and are separated by amino acid sequences that comprise scaffolding or framework regions. Among the various CDRs, the CDR3 sequences, and particularly CDRH3, are the most diverse and therefore have the strongest contribution to antibody specificity. There are at least two techniques for determining CDRs: (1) an approach based on cross-species sequence variability (i.e., Kabat et al., Sequences of Proteins of Immunological Interest (National Institute of Health, Bethesda, Md. (1987), incorporated by reference in its entirety); and (2) an approach based on crystallographic studies of antigen-antibody complexes (Chothia et al., Nature, 342:877 (1989), incorporated by reference in its entirety).

The term “epitope” means a protein determinant capable of specific binding to an antibody. Epitopes usually consist of chemically active surface groupings of molecules such as amino acids or sugar side chains. Certain epitopes can be defined by a particular sequence of amino acids to which an antibody is capable of binding. The term “extracellular epitope” refers to an epitope that is located on the outside of a cell's plasma membrane. Exemplary extracellular epitopes of plasma membrane expressed HCMV proteins are listed in Table 5.

As used herein, the term “humanized antibody” refers to an antibody that has at least one CDR derived from a mammal other than a human, and a FR region and the constant region of a human antibody.

As used herein, the term “monoclonal antibody” refers to an antibody obtained from a population of substantially homogeneous antibodies that specifically bind to the same epitope, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein).

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Target Proteins

In certain embodiments, provided herein are methods of treating HCMV infection by administering an agent (e.g., a therapeutic antibody) that specifically binds to an HCMV protein that is expressed on the plasma membrane of HCMV infected cells. In some embodiments the plasma membrane expressed HCMV protein is selected from among the proteins encoded by the genes listed in Table 1. In some embodiments, the agent binds to an extracellular epitope of a protein encoded by a gene listed in Table 1. The protein and gene reference numbers provided in Table 1 and elsewhere herein are merely exemplary and refer to the Merlin strain of HCMV. These protein and gene reference numbers are not meant to be limiting. The methods and compositions provided herein can be applied to any strain of HCMV. The corresponding gene and protein sequences of the genes listed in Table 1 in non-Merlin strains of HCMV are known in the art and/or readily determined without need for undue experimentation.

TABLE 1
Genes encoding selected HCMV proteins expressed
on the plasma membrane of HCMV infected cells.

| Gene | Uniport | GI Number | Description |
|---|---|---|---|
| UL142 | D2K3T4 | 395455117 | Membrane glycoprotein UL142 |
| UL9 | F5H9T4 | 384952364 | Membrane glycoprotein UL9 |
| UL1 | Q6SWC8 | 82013985 | Glycoprotein UL1 |
| UL5 | F5HHY9 | 82013982 | Protein UL5 |
| UL41A | F5HFG3 | 395455127 | Protein UL41A |
| RL12 | Q6SWD0 | 82013987 | Uncharacterized protein RL12 |
| UL33 | Q6SW98 | 82055331 | G-protein coupled receptor |
| | | | homolog UL33 |
| UL119 | F5HC14 | 391359343 | Viral Fc-gamma receptor-like |
| | | | protein UL119 |
| UL16 | F5HG68 | 395455121 | Protein UL16 |
| RL10 | F5HI32 | 395406822 | Protein IRL10 |
| UL100 | Q6SW43 | 82013927 | Envelope glycoprotein M |
| UL40 | Q6SW92 | 82013961 | Protein UL40 |
| US6 | Q6SW00 | 82013896 | Unique short US6 glycoprotein |
| UL144 | F5HAM0 | 363805602 | Membrane glycoprotein UL144 |
| US28 | Q80KM9 | 82058001 | Envelope protein US28 |
| US27 | F5HDK1 | 380875404 | Envelope glycoprotein US27 |
| RL11 | Q6SWD1 | 82013988 | Membrane glycoprotein RL11 |
| US9 | F5HC33 | 384951451 | Membrane glycoprotein US9 |
| UL148D | D2K3U5 | 77543601 | Protein UL148D |
| US20 | F5HGH8 | 395455141 | Membrane protein US20 |
| UL78 | B8YEA3 | 395455130 | Protein UL78 |
| UL136 | F5HF35 | 391359344 | Protein UL136 |
| US14 | F5HD92 | 384951455 | Membrane protein US14 |
| UL73 | F5HHQ0 | 380876918 | Envelope glycoprotein N |
| UL132 | D2K3S7 | 395455115 | Envelope glycoprotein UL132 |
| UL141 | Q6RJQ3 | 82013863 | Protein UL141 |
| UL14 | Q6SWB7 | 82013974 | Uncharacterized protein UL14 |
| UL22A | F5HF90 | 384952467 | Glycoprotein UL22A |
| US12 | F5HE44 | 395455137 | Uncharacterized protein US12 |
| UL103 | F5HA10 | 395455111 | Tegument protein UL103 |
| UL133 | Q6SW10 | 82013903 | Protein UL133 |
| US8 | F5HB52 | 384951444 | Membrane glycoprotein US8 |
| UL50 | Q6SW81 | 82013953 | Nuclear egress membrane protein |
| UL94 | F5HAC7 | 391359347 | Capsid-binding protein UL94 |
| UL13 | F5HGX4 | 82013975 | Protein UL13 |
| UL148 | F5H8Q3 | 395455119 | Membrane protein UL148 |
| UL99 | F5HI87 | 395455101 | Tegument protein UL99 |
| UL135 | F5HAQ7 | 384952459 | Protein UL135 |
| UL146 | F5HBX1 | 395406771 | Chemokine vCXCL1 |
| IRS1 | Q6SW04 | 82013899 | Protein IRS1 |
| UL44 | A9YU18 | 270355806 | DNA polymerase processivity |
| | | | factor |
| UL83 | Q6SW59 | 82013937 | 65 kDa phosphoprotein |

In certain embodiments, provided herein are methods of treating HCMV infection by administering an agent (e.g., a therapeutic antibody) that specifically binds to an HCMV protein that is expressed on the plasma membrane early after HCMV infection (e.g., within 24, 48 or 72 hours of HCMV infection). In some embodiments such early plasma membrane expressed HCMV protein is selected from among the proteins encoded by the genes listed in Table 2. In some embodiments, the agent binds to an extracellular epitope of a protein encoded by a gene listed in Table 2. The protein and gene reference numbers provided in Table 2 and elsewhere herein are merely exemplary and refer to the Merlin strain of HCMV. These protein and gene reference numbers are not meant to be limiting. The methods and compositions provided herein can be applied to any strain of HCMV. The corresponding gene and protein sequences of the genes listed in Table 2 in non-Merlin strains of HCMV are known in the art and/or readily determined without need for undue experimentation.

TABLE 2
Selected genes encoding selected HCMV proteins expressed on the plasma
membrane of HCMV infected cells soon after HCMV infection.

| Gene | Uniprot | GI Number | Description |
|---|---|---|---|
| UL9 | F5H9T4 | 384952364 | Membrane glycoprotein UL9 |
| UL5 | F5HHY9 | 82013982 | Protein UL5 |
| RL12 | Q6SWD0 | 82013987 | Uncharacterized protein RL12 |
| UL119 | F5HC14 | 391359343 | Viral Fc-gamma receptor-like |
| | | | protein UL119 |
| UL16 | F5HG68 | 395455121 | Protein UL16 |
| UL40 | Q6SW92 | 82013961 | Protein UL40 |
| US6 | Q6SW00 | 82013896 | Unique short US6 glycoprotein |
| US28 | Q80KM9 | 82058001 | Envelope protein US28 |
| RL11 | Q6SWD1 | 82013988 | Membrane glycoprotein RL11 |
| US9 | F5HC33 | 384951451 | Membrane glycoprotein US9 |
| UL148D | D2K3U5 | 77543601 | Protein UL148D |
| US20 | F5HGH8 | 395455141 | Membrane protein US20 |
| UL78 | B8YEA3 | 395455130 | Protein UL78 |
| UL136 | F5HF35 | 391359344 | Protein UL136 |
| US14 | F5HD92 | 384951455 | Membrane protein US14 |
| UL14 | Q6SWB7 | 82013974 | Uncharacterized protein UL14 |
| US12 | F5HE44 | 395455137 | Uncharacterized protein US12 |
| UL103 | F5HA10 | 395455111 | Tegument protein UL103 |
| UL133 | Q6SW10 | 82013903 | Protein UL133 |
| US8 | F5HB52 | 384951444 | Membrane glycoprotein US8 |
| UL13 | F5HGX4 | 82013975 | Protein UL13 |
| UL135 | F5HAQ7 | 384952459 | Protein UL135 |
| IRS1 | Q6SW04 | 82013899 | Protein IRS1 |

In some embodiments, provided herein are methods of treating HCMV infection by administering an agent (e.g., a therapeutic antibody) that specifically binds to an endogenous protein that is upregulated on the plasma membrane after HCMV infection. In some embodiments, the endogenous protein is upregulated at the plasma membrane soon after HCMV infection (e.g., within 24, 48 or 72 hours of HCMV infection). In some embodiments the endogenous protein is selected from among the proteins encoded by the genes listed in Table 3 or Table 4. In some embodiments, the agent binds to an extracellular epitope of a protein encoded by a gene listed in Table 3 or Table 4.

TABLE 3
Genes encoding selected endogenous proteins upregulated on the plasma
membrane of HCMV infected cells after HCMV infection.

| Gene | | GI | |
|---|---|---|---|
| Symbol | Uniprot | Number | Protein name |
| CHST11 | Q9NPF2 | 61212137 | Carbohydrate sulfotransferase 11 |
| KCNK1 | O00180 | 13124036 | Potassium channel subfamily K member 1 |
| SPINT1 | O43278 | 61252335 | Kunitz-type protease inhibitor 1 |
| CDH1 | P12830 | 399166 | Cadherin-1 |
| CEACAM1 | P13688 | 399116 | Carcinoembryonic antigen-related cell adhesion |
| | | | molecule 1 |
| EPCAM | P16422 | 160266056 | Epithelial cell adhesion molecule |
| TNFRSF1B | P20333 | 21264534 | Tumor necrosis factor receptor superfamily |
| | | | member 1B |
| ERBB3 | P21860 | 119534 | Receptor tyrosine-protein kinase erbB-3 |
| CNTFR | P26992 | 1352099 | Ciliary neurotrophic factor receptor subunit |
| | | | alpha |
| PCDH1 | Q08174 | 215273864 | Protocadherin-1 |
| BST2 | Q10589 | 1705508 | Bone marrow stromal antigen 2 |
| SDK2 | Q58EX2 | 296452966 | Protein sidekick-2 |
| RALGPS2 | Q86X27 | 74750518 | Ras-specific guanine nucleotide-releasing factor |
| | | | RalGPS2 |
| SLCO4A1 | Q96BD0 | 27734555 | Solute carrier organic anion transporter family |
| | | | member 4A1 |
| MEGF10 | Q96KG7 | 74716908 | Multiple epidermal growth factor-like domains |
| | | | protein 10 |
| SEMA4D | Q92854 | 8134701 | Semaphorin-4D |
| PCDH1 | Q08174 | 215273864 | Protocadherin-1 |
| SPINT1 | O43278 | 61252335 | Kunitz-type protease inhibitor 1 |
| TTC17 | Q96AE7 | 52783467 | Tetratricopeptide repeat protein 17 |
| MFSD2A | Q8NA29 | 74751132 | Major facilitator superfamily domain-containing |
| | | | protein 2A |
| DNAH1 | Q9P2D7 | 327478598 | Dynein heavy chain 1, axonemal |
| GFRA2 | O00451 | 118582303 | GDNF family receptor alpha-2 |
| P2RY2 | P41231 | 311033490 | P2Y purinoceptor 2 |
| TYRO3 | Q06418 | 1717829 | Tyrosine-protein kinase receptor TYRO3 |
| TSPAN18 | Q96SJ8 | 68053316 | Tetraspanin-18 |
| SLC38A3 | Q99624 | 52783419 | Sodium-coupled neutral amino acid transporter 3 |
| CADM1 | Q9BY67 | 150438862 | Cell adhesion molecule 1 |
| RTN4R | Q9BZR6 | 25453267 | Reticulon-4 receptor |
| SLC39A8 | Q9C0K1 | 74733496 | Zinc transporter ZIP8 |
| NPDC1 | Q9NQX5 | 22261810 | Neural proliferation differentiation and control |
| | | | protein 1 |
| CACNA2D2 | Q9NY47 | 387912827 | Voltage-dependent calcium channel subunit |
| | | | alpha-2/delta-2 |
| PODXL2 | Q9NZ53 | 74734719 | Podocalyxin-like protein 2 |
| NPC1L1 | Q9UHC9 | 425906049 | Niemann-Pick C1-like protein 1 |
| SLC7A8 | Q9UHI5 | 12643348 | Large neutral amino acids transporter small |
| | | | subunit 2 |
| LIFR | P42702 | 1170784 | Leukemia inhibitory factor receptor |
| NCAM1 | P13591 | 205830665 | Neural cell adhesion molecule 1 |
| MMP15 | P51511 | 1705988 | Matrix metalloproteinase-15 |
| NGFR | P08138 | 128156 | Tumor necrosis factor receptor superfamily |
| | | | member 16 |
| SCARB1 | Q8WTV0 | 37999904 | Scavenger receptor class B member 1 |
| CD55 | P08174 | 60416353 | Complement decay-accelerating factor |
| GPR108 | Q9NPR9 | 296439338 | Protein GPR108 |
| HLA-E | P13747 | 34395942 | HLA class I histocompatibility antigen, alpha |
| | | | chain E |
| F11R | Q9Y624 | 10720061 | Junctional adhesion molecule A |
| GPR56 | Q9Y653 | 45476992 | G-protein coupled receptor 56 |
| ERO1LB | Q86YB8 | 116241353 | ERO1-like protein beta |
| B3GNT9 | Q6UX72 | 74738184 | UDP-GlcNAc:betaGal beta-1,3-N- |
| | | | acetylglucosaminyltransferase 9 |
| ERO1L | Q96HE7 | 50400608 | ERO1-like protein alpha |
| SREK1 | Q8WXA9 | 37537968 | Splicing regulatory glutamine/lysine-rich |
| | | | protein 1 |
| IQGAP2 | Q13576 | 37537968 | Ras GTPase-activating-like protein IQGAP2 |
| TSPAN13 | O95857 | 11135162 | Tetraspanin-13 |
| PRICKLE2 | Q7Z3G6 | 85701877 | Prickle-like protein 2 |
| ABCA3 | Q99758 | 85700402 | ATP-binding cassette sub-family A member 3 |
| SLC27A6 | Q9Y2P4 | 74725713 | Long-chain fatty acid transport protein 6 |
| LUC7L3 | O95232 | 94730369 | Luc7-like protein 3 |
| HSPA9 | P38646 | 21264428 | Stress-70 protein, mitochondrial |
| PTGS2 | P35354 | 3915797 | Prostaglandin G/H synthase 2 |
| C19orf10 | Q969H8 | 61221730 | UPF0556 protein C19orf10 |
| HSPA5 | P11021 | 14916999 | 78 kDa glucose-regulated protein |
| CCDC134 | Q9H6E4 | 74752694 | Coiled-coil domain-containing protein 134 |
| ARHGAP31 | Q2M1Z3 | 296452881 | Rho GTPase-activating protein 31 |
| CRELD1 | Q96HD1 | 209572751 | Isoform 2 of Cysteine-rich with EGF-like |
| | | | domain protein 1 |
| PSAP | P07602 | 134218 | Proactivator polypeptide |
| CERCAM | Q5T4B2 | 74744901 | Glycosyltransferase 25 family member 3 |
| ARHGAP21 | Q5T5U3 | 74745129 | Rho GTPase-activating protein 21 |
| MCFD2 | Q8NI22 | 49036425 | Multiple coagulation factor deficiency protein 2 |
| GNB2L1 | P63244 | 54037168 | Guanine nucleotide-binding protein subunit |
| | | | beta-2-like 1 |
| DST | Q03001 | 294862529 | Dystonin |
| HSPA13 | P48723 | 1351125 | Heat shock 70 kDa protein 13 |
| B3GNT2 | Q9NY97 | 29840874 | UDP-GlcNAc:betaGal beta-1,3-N- |
| | | | acetylglucosaminyltransferase 2 |
| VPS13D | Q5THJ4 | 74756617 | Vacuolar protein sorting-associated protein 13D |
| SLC39A7 | Q92504 | 12643344 | Zinc transporter SLC39A7 |
| SRRM1 | Q8IYB3 | 83305833 | Serine/arginine repetitive matrix protein 1 |
| HSPA1A | P08107 | 147744565 | Heat shock 70 kDa protein 1A/1B |
| TOR1B | O14657 | 13878818 | Torsin-1B |
| GRPEL1 | Q9HAV7 | 18202951 | GrpE protein homolog 1, mitochondrial |
| PRPF4B | Q13523 | 317373526 | Serine/threonine-protein kinase PRP4 homolog |
| TBCEL | Q5QJ74 | 215273924 | Tubulin-specific chaperone cofactor E-like |
| | | | protein |
| RSRC2 | Q7L4I2 | 74739167 | Arginine/serine-rich coiled-coil protein 2 |
| BAG3 | O95817 | 12643665 | BAG family molecular chaperone regulator 3 |
| IFIT2 | P09913 | 124488 | Interferon-induced protein with tetratricopeptide |
| | | | repeats 2 |
| BRD4 | O60885 | 20141192 | Bromodomain-containing protein 4 |
| HYOU1 | Q9Y4L1 | 10720185 | Hypoxia up-regulated protein 1 |

TABLE 4
Preferred genes encoding selected endogenous proteins upregulated on the plasma
membrane of HCMV infected cells after HCMV infection.

| Gene | | GI | |
|---|---|---|---|
| Symbol | Uniprot | Number | Protein name |
| CHST11 | Q9NPF2 | 61212137 | Carbohydrate sulfotransferase 11 |
| KCNK1 | O00180 | 13124036 | Potassium channel subfamily K member 1 |
| SPINT1 | O43278 | 61252335 | Kunitz-type protease inhibitor 1 |
| CDH1 | P12830 | 399166 | Cadherin-1 |
| CEACAM1 | P13688 | 399116 | Carcinoembryonic antigen-related cell adhesion |
| | | | molecule 1 |
| EPCAM | P16422 | 160266056 | Epithelial cell adhesion molecule |
| TNFRSF1B | P20333 | 21264534 | Tumor necrosis factor receptor superfamily |
| | | | member 1B |
| ERBB3 | P21860 | 119534 | Receptor tyrosine-protein kinase erbB-3 |
| CNTFR | P26992 | 1352099 | Ciliary neurotrophic factor receptor subunit |
| | | | alpha |
| PCDH1 | Q08174 | 215273864 | Protocadherin-1 |
| BST2 | Q10589 | 1705508 | Bone marrow stromal antigen 2 |
| SDK2 | Q58EX2 | 296452966 | Protein sidekick-2 |
| RALGPS2 | Q86X27 | 74750518 | Ras-specific guanine nucleotide-releasing factor |
| | | | RalGPS2 |
| SLCO4A1 | Q96BD0 | 27734555 | Solute carrier organic anion transporter family |
| | | | member 4A1 |
| MEGF10 | Q96KG7 | 74716908 | Multiple epidermal growth factor-like domains |
| | | | protein 10 |
| SEMA4D | Q92854 | 8134701 | Semaphorin-4D |
| PCDH1 | Q08174 | 215273864 | Protocadherin-1 |
| SPINT1 | O43278 | 61252335 | Kunitz-type protease inhibitor 1 |
| TTC17 | Q96AE7 | 52783467 | Tetratricopeptide repeat protein 17 |

### Antibodies

In certain embodiments, the compositions and methods provided herein relate to antibodies and antigen binding fragments thereof that bind specifically to a protein expressed on the plasma membrane of an HCMV infected cell (e.g., a protein encoded by a gene listed in Tables 1-4). In some embodiments, the antibodies bind to a particular epitope of one of the target proteins provided herein. In some embodiment the epitope is an extracellular epitope. In some embodiments, the epitope is an epitope listed in Table 5. In some embodiments, the antibodies can be polyclonal or monoclonal and can be, for example, murine, chimeric, humanized or fully human.

TABLE 5
Exemplary extracellular epitopes of
plasma membrane expressed HCMV proteins.

| | First | Last | |
|---|---|---|---|
| Gene | Amino | Amino | |
| Symbol | Acid | Acid | Epitope Sequence |
| UL9 | 6 | 16 | MTIPCTPTVGY |
| UL9 | 18 | 28 | SHNISLHPLNN |
| UL9 | 45 | 52 | VTNKLCLY |
| UL9 | 87 | 102 | SRNYYFQSFKYLGQGV |
| UL9 | 104 | 143 | KPNNLCYNVSVHFTHQTHCHTTTSSLY |
| | | | PPTSVHDSLEISQ |
| UL9 | 151 | 164 | THTAVHYAAGNVEA |
| UL5 | 23 | 40 | AFTSSVSTRTPSLAIAPP |
| UL5 | 50 | 63 | EEELVPWSRLIITK |
| RL12 | 13 | 29 | YRQTVYIILTFYIVYRG |
| RL12 | 47 | 56 | VSDTSVYSTP |
| RL12 | 106 | 114 | TASTLTALS |
| RL12 | 157 | 170 | TYSPVTSIAVNCTV |
| RL12 | 188 | 194 | GTIRVKS |
| RL12 | 214 | 221 | NCPNVVWY |
| RL12 | 228 | 235 | THGHHIYP |
| RL12 | 240 | 271 | QTPTYQHKILTSHPICHPDVSSPAAYH |
| | | | DLCRS |
| RL12 | 290 | 296 | YSRRCYK |
| RL12 | 323 | 332 | TTPLCPRYVG |
| U1119 | 25 | 36 | NVSSAVTTTVQT |
| U1119 | 41 | 47 | ASTSVIA |
| U1119 | 52 | 80 | EGHLYTVNCEASYSYDQVSLNATCKVI |
| | | | LL |
| U1119 | 86 | 96 | PDILSVTCYAR |
| U1119 | 99 | 111 | CKGPFTQVGYLSA |
| U1119 | 118 | 125 | GKLHLSYN |
| U1119 | 128 | 135 | AQELLISG |
| U1119 | 142 | 148 | TEYTCSF |
| U1119 | 160 | 171 | DLFTYPIYAVYG |
| U1119 | 179 | 216 | MRVRVLLQEHEHCLLNGSSLYHPNSTV |
| | | | HLHQGDQLIPP |
| U1119 | 229 | 250 | LREFVFYLNGTYTVVRLHVQIA |
| U1119 | 255 | 264 | TTTYVFIKSD |
| UL16 | 13 | 27 | SNSTCRLNVTELASI |
| UL16 | 35 | 46 | LHGMCISICYYE |
| UL16 | 52 | 58 | EIIGVAF |
| UL16 | 62 | 71 | HNESVVDLWL |
| UL16 | 94 | 103 | KMRTVPVTKL |
| UL16 | 113 | 121 | TVGRYDCLR |
| UL16 | 129 | 143 | IIERLYVRLGSLYPR |
| UL16 | 145 | 157 | PGSGLAKHPSVSA |
| UL40 | 10 | 38 | TTAGVTSAHGPLCPLVFQGWAYAVYHQ |
| | | | GD |
| UL40 | 40 | 51 | VLMTLDVYCCRQ |
| UL40 | 53 | 62 | SSNTVVAFSH |
| UL40 | 65 | 72 | ADNTLLIE |
| UL40 | 80 | 106 | HVDGISCQDHFRAQHQDCPAQTVHVRG |
| UL40 | 111 | 142 | AFGLTHLQSCCLNEHSQLSERVAYHLK |
| | | | LRPAT |
| UL40 | 149 | 181 | AMYTVGILALGSFSSFYSQIARSLGVL |
| | | | PNDHHY |
| US6 | 7 | 22 | PKTLLSLSPRQACVPR |
| US6 | 25 | 31 | SHRPVCY |
| US6 | 51 | 58 | FAHQCLQA |
| US6 | 77 | 111 | GRLTCQRVRRLLPCDLDIHPSHRLLTL |
| | | | MNNCVCDG |
| US6 | 113 | 119 | VWNAFRL |
| RL11 | 10 | 20 | KKPLKLANYRA |
| RL11 | 26 | 32 | TRTLVTR |
| RL11 | 34 | 49 | NTSHHSVVWQRYDIYS |
| RL11 | 55 | 62 | MPPLCIIT |
| RL11 | 82 | 100 | NLTLYNLTVKDTGVYLLQD |
| RL11 | 102 | 121 | YTGDVEAFYLIIHPRSFCRA |
| RL11 | 123 | 139 | ETRRCFYPGPGRVVVTD |
| US9 | 17 | 26 | SSSRICPLSN |
| US9 | 28 | 35 | KSVRLPQY |
| US9 | 41 | 68 | DVSGYRVSSSVSECYVQHGVLVAAWLV |
| | | | R |
| US9 | 89 | 95 | THFKVGA |
| US9 | 108 | 152 | TELPQVDARLSYVMLTVYPCSACNRSV |
| | | | LHCRPASRLPWLPLRVTP |
| UL78 | 4 | 13 | VLRGVLQPAS |
| UL78 | 21 | 30 | IMDYVELATR |
| UL78 | 33 | 48 | LTMRLGILPLFIIAFF |
| UL78 | 58 | 127 | DSFDYLVERCQQSCHGHFVRRLVQALK |
| | | | RAMYSVELAVCYFSTSVRDVAEAVKKS |
| | | | SSRCYADATSAAVVVT |
| UL78 | 149 | 164 | PGTTIDVSAESSSVLC |
| UL136 | 13 | 29 | MLHDLFCGCHYPEKCRR |
| UL136 | 62 | 68 | YGSGCRF |
| UL136 | 79 | 85 | PAPPALS |
| UL136 | 125 | 142 | DAVHVAVQAAVQATVQVS |
| US14 | 7 | 21 | MFSYLAKLGTYHHYR |
| US15 | 24 | 32 | NGTLSVILN |
| UL14 | 4 | 15 | APPVVRSPCLQP |
| UL14 | 26 | 33 | GSPQLLPY |
| UL14 | 35 | 45 | DRLEVACIFPA |
| UL14 | 47 | 85 | DWPEVSIRVHLCYWPEIVRSLVVDARS |
| | | | GQVLHNDASCYI |
| UL14 | 97 | 109 | AAQRLSLSFRLIT |
| UL14 | 113 | 120 | GTYTCVLG |
| UL14 | 130 | 140 | TTALVADVHDL |
| UL14 | 143 | 151 | SDRSCDLAF |
| UL14 | 156 | 162 | QTRYLWT |
| UL14 | 179 | 195 | RHRVVHYIPGTSGLLPS |
| UL14 | 201 | 210 | RELCVPFISQ |
| UL14 | 228 | 234 | RRYHLRR |
| UL103 | 5 | 14 | MIRGVLEVHT |
| UL103 | 23 | 31 | IMEPQVLDF |
| UL103 | 42 | 50 | TEHGLLVSM |
| UL103 | 53 | 74 | YRSELLCTSAFLGYSAVFLLET |
| UL103 | 77 | 114 | AVTQVRLSDLRLKHRCGIVKADNLLHF |
| | | | ALCTVISCVEN |
| UL103 | 117 | 134 | LTRKCLHDLLQYLDAVNV |
| UL103 | 138 | 158 | FGRLLHHSARRLICSALYLLF |
| UL103 | 162 | 177 | EPHIVQYVPATFVLFQ |
| UL103 | 179 | 193 | TRHTCLQLVARFFFR |
| UL103 | 199 | 206 | EAHSFSLK |
| UL103 | 214 | 227 | DGWPVGLGLLDVLN |
| UL103 | 230 | 239 | YPNLPSPPKL |
| UL103 | 230 | 239 | YPNLPSPPKL |
| US8 | 22 | 35 | EPNYVAPPARQFRF |
| US8 | 37 | 63 | PLNNVSSYQASCVVKDGVLDAVWRVQG |
| US8 | 67 | 74 | PEKGIVAR |
| US8 | 87 | 124 | RLHAPECLVETTEAVFRLRQWVPTDLD |
| | | | HLTLHLVPCTK |
| US8 | 126 | 138 | KPMWCQPRYHIRY |
| UL13 | 14 | 25 | QGATYQLSIVRQ |
| UL13 | 30 | 38 | AGFQVRAAS |
| UL13 | 44 | 85 | NAVDLDRPPLWSGSLPHLPVYDVRSPR |
| | | | PLRPPSSQHHAVSPE |
| UL13 | 95 | 104 | QYQELQYLVE |
| UL13 | 116 | 128 | IPRPSFPPPDPPS |
| UL13 | 148 | 154 | AESTVSH |
| UL13 | 177 | 185 | SRDSLLLTR |
| UL13 | 218 | 246 | GLRQLRQQLTVRWQLFRLRCHGWTQQV |
| | | | SS |
| UL13 | 254 | 262 | ESNVVSQTA |
| UL13 | 266 | 272 | RTWFVQR |
| UL13 | 289 | 303 | EAQELAIIPPAPTVL |
| UL13 | 364 | 372 | EVQEPQVTY |
| UL13 | 401 | 410 | NTLTVACPPR |
| UL13 | 413 | 432 | PHRALFRLCLGLWVSSYLVR |
| IRS1 | 24 | 37 | SGVGSSPPSSCVPM |
| IRS1 | 55 | 62 | PGHGVHRV |
| IRS1 | 84 | 96 | PERLLLSQIPVER |
| IRS1 | 98 | 104 | ALTELEY |
| IRS1 | 110 | 116 | VWRAAFL |
| IRS1 | 132 | 150 | AGTLLPLGRPYGFYARVTP |
| IRS1 | 169 | 184 | DAWIVLVATVVHEVDP |
| IRS1 | 196 | 220 | HPEGLCAQDGLYLALGAGFRVFVYD |
| IRS1 | 223 | 230 | NNTLILAA |
| IRS1 | 240 | 252 | GAGEVVRLYRCNR |
| IRS1 | 259 | 274 | RATLLPQPALRQTLLR |
| IRS1 | 291 | 297 | GTTVALQ |
| IRS1 | 303 | 336 | LQPMVLLGAWQELAQYEPFASAPHPAS |
| | | | LLTAVRR |
| IRS1 | 338 | 362 | LNQRLCCGWLALGAVLPARWLGCAA |
| IRS1 | 384 | 404 | GDAPCAMAGAVGSAVTIPPQP |
| IRS1 | 410 | 426 | GSAICVPNADAHAVVGA |
| IRS1 | 428 | 443 | ATAAAAAAAAAPTVMV |
| IRS1 | 458 | 503 | PRAMLVVVLDELGAVFGYCPLDGHVYP |
| | | | LAAELSHFLRAGVLGALAL |
| IRS1 | 513 | 520 | AARRLLPE |
| IRS1 | 531 | 544 | WDALHLHPRAALWA |
| IRS1 | 563 | 571 | IHDPVAFRL |
| IRS1 | 575 | 583 | RTLGLDLTT |
| IRS1 | 589 | 602 | QSQLPEKYIGFYQI |
| IRS1 | 625 | 640 | TMPPPLSAQASVSYAL |
| IRS1 | 648 | 655 | RPLSTVDD |
| IRS1 | 664 | 670 | ESHWVLG |
| IRS1 | 695 | 706 | RPMPVVPEECYD |
| IRS1 | 712 | 722 | EGHQVIPLCAS |
| IRS1 | 749 | 756 | KPPRLCKT |
| IRS1 | 759 | 765 | GPPPLPP |
| IRS1 | 833 | 842 | RPKKCQTHAP |

Polyclonal antibodies can be prepared by immunizing a suitable subject (e.g. a mouse) with a polypeptide immunogen (e.g., a protein encoded by a gene listed in Tables 1-4 or a fragment thereof). In some embodiments, the polypeptide immunogen comprises an extracellular epitope of a target protein provided herein. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well known techniques, such as protein A chromatography to obtain the IgG fraction.

At an appropriate time after immunization, e.g., when the antibody titers are highest, antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), a human B cell hybridoma technique (Kozbor et al. (1983) Immunol. Today 4:72), a EBV-hybridoma technique (Cole et al. (1985) Monoclonal Antibodies and Cancer Therapy, Alan R. Liss, Inc., pp. 77-96) or a trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

As an alternative to preparing monoclonal antibody-secreting hybridomas, a monoclonal antibody that binds to a target protein described herein can be obtained by screening a recombinant combinatorial immunoglobulin library (e.g., an antibody phage display library or an antibody yeast display library) with the appropriate polypeptide (e.g. a polypeptide comprising an extracellular epitope of a target protein described herein) to thereby isolate immunoglobulin library members that bind the polypeptide.

Additionally, recombinant antibodies specific for a target protein provided herein and/or an extracellular epitope of a target protein provided herein, such as chimeric or humanized monoclonal antibodies, can be made using standard recombinant DNA techniques. Such chimeric and humanized monoclonal antibodies can be produced by recombinant DNA techniques known in the art, for example using methods described in U.S. Pat. No. 4,816,567; U.S. Pat. No. 5,565,332; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Human monoclonal antibodies specific for a target protein provided herein and/or an extracellular epitope of a target protein provided herein can be generated using transgenic or transchromosomal mice carrying parts of the human immune system rather than the mouse system. For example, “HuMAb mice” which contain a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al. (1994) Nature 368(6474): 856 859). Accordingly, the mice exhibit reduced expression of mouse IgM or κ, and in response to immunization, the introduced human heavy and light chain transgenes undergo class switching and somatic mutation to generate high affinity human IgGic monoclonal antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N. Y Acad. Sci 764:536 546). The preparation of HuMAb mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807.

In certain embodiments, the antibodies provided herein are able to bind to an epitope of a protein encoded by a gene listed in Tables 1-4 (e.g., an extracellular epitope) with a dissociation constant of no greater than 10⁻⁶, 10⁻⁷, 10⁻⁸ or 10⁻⁹ M. Standard assays to evaluate the binding ability of the antibodies are known in the art, including for example, ELISAs, Western blots and RIAs. The binding kinetics (e.g., binding affinity) of the antibodies also can be assessed by standard assays known in the art, such as by Biacore analysis.

In some embodiments the antibody is part of an antibody-drug conjugate. Antibody-drug conjugates are therapeutic molecules comprising an antibody (e.g., an antibody that binds to a protein encoded by a gene listed in Tables 1-4) linked to a biologically active agent, such as a cytotoxic agent or an antiviral agent. In some embodiments, the biologically active agent is linked to the antibody via a chemical linker. Such linkers can be based on any stable chemical motif, including disulfides, hydrazones, peptides or thioethers. In some embodiments, the linker is a cleavable linker and the biologically active agent is released from the antibody upon antibody binding to the plasma membrane target protein. In some embodiments, the linker is a noncleavable linker.

In some embodiments, the antibody-drug conjugate comprises an antibody linked to a cytotoxic agent. In certain embodiments, any cytotoxic agent able to kill HCMV infected cells can be used. In some embodiments, the cytotoxic agent is MMAE, DM-1, a maytansinoid, a doxorubicin derivative, an auristatin, a calcheamicin, CC-1065, an aduocarmycin or an anthracycline.

In some embodiments, the antibody-drug conjugate comprises an antibody linked to an antiviral agent. In some embodiments, any antiviral agent capable of inhibiting HCMV replication is used. In some embodiments, the antiviral agent is ganciclovir, valganciclovir, foscarnet, cidofovir, acyclovir, formivirsen, maribavir, BAY 38-4766 or GW275175X.

### Nucleic Acid Molecules

Provided herein are nucleic acid molecules that encode the antibodies described herein. The nucleic acids may be present, for example, in whole cells, in a cell lysate, or in a partially purified or substantially pure form.

Nucleic acid molecules provided herein can be obtained using standard molecular biology techniques. For example, nucleic acid molecules described herein can be cloned using standard PCR techniques or chemically synthesized. For nucleic acids encoding antibodies expressed by hybridomas, cDNAs encoding the light and/or heavy chains of the antibody made by the hybridoma can be obtained by standard PCR amplification or cDNA cloning techniques. For antibodies obtained from an immunoglobulin gene library (e.g., using phage or yeast display techniques), nucleic acid encoding the antibody can be recovered from the library.

Once DNA fragments encoding a V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

In certain embodiments, provided herein are vectors that contain the isolated nucleic acid molecules described herein. As used herein, the term “vector,” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”).

In certain embodiments, provided herein are cells that contain a nucleic acid described herein (e.g., a nucleic acid encoding an antibody, antigen binding fragment thereof or polypeptide described herein). The cell can be, for example, prokaryotic, eukaryotic, mammalian, avian, murine and/or human. In certain embodiments the cell is a hybridoma. In certain embodiments the nucleic acid provided herein is operably linked to a transcription control element such as a promoter. In some embodiments the cell transcribes the nucleic acid provided herein and thereby expresses an antibody, antigen binding fragment thereof or polypeptide described herein. The nucleic acid molecule can be integrated into the genome of the cell or it can be extrachromasomal.

### Therapeutic Agents

In certain embodiments, provided herein are methods and compositions for treating HCMV by administering to a subject an agent that binds to a target protein provided herein (e.g., a protein encoded by a gene listed in Tables 1-4). Agents which may be used to for the methods provided herein include antibodies (e.g., an antibody described herein), proteins, peptides and small molecules.

In some embodiments, any agent that binds to a target protein provided herein can be used to practice the methods described herein. Such agents can be those described herein, those known in the art, or those identified through routine screening assays (e.g. the screening assays described herein).

In some embodiments, assays used to identify agents useful in the methods described herein include a reaction between a target protein provided herein or fragment thereof and a test compound (e.g. the potential agent). Agents useful in the methods described herein may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994)J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

Agents useful in the methods provided herein can be identified, for example, using assays for screening candidate or test compounds which are able to bind to a target protein provided herein or a fragment thereof. The basic principle of the assay systems used to identify compounds that bind to a target protein provided herein or fragment thereof involves preparing a reaction mixture containing the target protein or fragment thereof and a test agent. The formation of any complexes between the target protein or fragment thereof and the test agent is then detected and test compounds that are able to specifically bind to the target protein or fragment thereof are identified as potential therapeutic agents. Such assays can be conducted in a heterogeneous or homogeneous format. Heterogeneous assays involve anchoring either the target protein or the test compound onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested.

In a heterogeneous assay system, either the target protein or the test agent is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of target protein or test agent and drying. Alternatively, an immobilized antibody specific for the assay component to be anchored can be used for this purpose.

In related assays, a fusion protein can be provided which adds a domain that allows one or both of the assay components to be anchored to a matrix. For example, glutathione-S-transferase/marker fusion proteins or glutathione-S-transferase/binding partner can be adsorbed onto glutathione sepharose beads (Sigma Chemical, St. Louis, Mo.) or glutathione derivatized microtiter plates can be used. Following incubation, the beads or microtiter plate wells are washed to remove any unbound assay components, the immobilized complex assessed either directly or indirectly, for example, as described above.

A homogeneous assay may also be used to identify agents that bind to a target protein or fragment thereof. This is typically a reaction, analogous to those mentioned above, which is conducted in a liquid phase. The formed complexes are then separated from unreacted components, and the amount of complex formed is determined.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August; 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et at (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads.

### Pharmaceutical Compositions

In certain embodiments provided herein is a composition, e.g., a pharmaceutical composition, containing at least one agent described herein (e.g., an antibody described herein) formulated together with a pharmaceutically acceptable carrier. In one embodiment, the composition includes a combination of multiple (e.g., two or more) agents provided herein.

The pharmaceutical compositions provided herein can be administered in combination therapy, i.e., combined with other agents. For example, the pharmaceutical composition also include an anti-viral drug that inhibits HCMV replication, such as, ganciclovir, valganciclovir, foscarnet, cidofovir, acyclovir, formivirsen, maribavir, BAY 38-4766 or GW275175X.

The pharmaceutical compositions provided herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions provided herein suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, agents provided herein, which may be used in a suitable hydrated form, and/or the pharmaceutical compositions of the provided herein, are formulated into pharmaceutically-acceptable dosage forms by conventional methods known to those of skill in the art.

### Therapeutic Methods

Disclosed herein are novel therapeutic methods of treatment or prevention of HCMV infection. In some embodiments, the methods provided herein comprise administering to a subject, (e.g., a subject in need thereof), an effective amount of an agent (e.g., an antibody) that binds to a target protein provided herein (e.g., a protein encoded by a gene listed in Tables 1-4). The compositions provided herein may be delivered by any suitable route of administration.

In some embodiments, the subject is a subject is susceptible to HCMV infection. In some embodiments, the subject in need thereof is immunocompromised. In some embodiments, the subject is HIV-infected or has AIDS. In some embodiments, the subject is an organ transplant recipient. In some embodiments, the subject is a bone marrow transplant recipient. In some embodiments, the subject is a newborn infant or is pregnant. In some embodiments, the subject has multiple myeloma, chronic lymphoid leukemia. In some embodiments the subject has undergone chemotherapy. In some embodiments, the subject has undergone immunosuppressive therapy.

In some embodiments, the agents provided herein can be administered in combination therapy, i.e., combined with other agents. For example, an agent provided herein can be administered as part of a conjunctive therapy in combination with an anti-viral drug that inhibits HCMV replication, such as, ganciclovir, valganciclovir, foscarnet, cidofovir, acyclovir, formivirsen, maribavir, BAY 38-4766 or GW275175X.

Conjunctive therapy includes sequential, simultaneous and separate, and/or co-administration of the active compounds in a such a way that the therapeutic effects of the first agent administered have not entirely disappeared when the subsequent agent is administered. In certain embodiments, the second agent may be co-formulated with the first agent or be formulated in a separate pharmaceutical composition.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds provided herein employed in the pharmaceutical composition at levels lower than that required in order to achieve the desired therapeutic effect and gradually increase the dosage until the desired effect is achieved.

### Examples

The invention now being generally described will be more readily understood by reference to the following examples which are included merely for purposes of illustration of certain aspects and embodiments of the present invention, and are not intended to limit the invention in any way.

### Experimental Procedures

### Cells and Viruses

Primary human fetal foreskin fibroblast cells (HFFF) were grown in Dulbecco's modified eagles medium (DMEM) (Life Technologies) supplemented with fetal bovine serum (10% v/v), penicillin/streptomycin and L-glutamine (Gibco) at 37° C. in 5% CO2. Cells were verified to be mycoplasma negative.

The HCMV strain Merlin is designated the reference HCMV genome sequence by the National Center for Biotechnology Information and was sequenced after only 3 passages in vitro. A BAC clone containing the complete Merlin genome was constructed to provide a reproducible source of genetically intact, clonal virus for pathogenesis studies (Stanton et al., J. Clin. Invest. 120:3191-3208 (2010), hereby incorporated by reference). Merlin BAC derived clone RCMV 1111 used herein contains point mutations in RL13 and UL128, enhancing replication in fibroblasts.

### Virus Infection

Twenty-four hours prior to each infection, 1.5×10⁷ HFFFs were plated in a 150 cm² flask. Cells were sequentially infected at multiplicity of infection 10 with HCMV strain Merlin. Infections were staggered such that all flasks were harvested simultaneously.

### Plasma Membrane Profiling (PMP)

PMP was performed as described in Weekes et al., J. Proteome. Res. 11:1475-1480 (2012) and Weekes et al., J. Biomol. Tech. 21:108-115 (2010), each of which is incorporated by reference in its entirety, with minor modifications for adherent cells. Briefly, one 150 cm² flask of HCMV-infected HFFFs per condition was washed twice with ice-cold PBS. Sialic acid residues were oxidized with sodium meta-periodate (Thermo) then biotinylated with aminooxy-biotin (Biotium). The reaction was quenched, and the biotinylated cells scraped into 1% Triton X-100 lysis buffer. Biotinylated glycoproteins were enriched with high affinity streptavidin agarose beads (Pierce) and washed extensively. Captured protein was denatured with DTT, alkylated with iodoacetamide (IAA, Sigma) and digested on-bead with trypsin (Promega) in 100 mM HEPES pH 8.5 for 3 hours. Tryptic peptides were then collected.

### Preparation of Whole Proteome Samples

Cells were washed twice with PBS, and 1 ml lysis buffer added (experiment 1: 8M Urea/100 mM HEPES pH8.5, experiment 2: 6M Guanidine/50 mM HEPES pH8.5). Cell lifters (Corning) were used to scrape cells in lysis buffer, which was removed to an eppendorf tube, vortexed extensively then sonicated. Cell debris was removed by centrifugation. Dithiothreitol (DTT) was added to a final concentration of 5 mM and samples were incubated for 20 minutes. Cysteines were alkylated by exposure to 15 mM iodoacetamide for 20 minutes in the dark. Excess iodoacetamide was quenched with DTT for 15 minutes. Samples were diluted with 100 mM HEPES pH 8.5 to 4M Urea or 1.5M Guanidine followed by digestion at room temperature for 3 hours with LysC protease at a 1:100 protease-to-protein ratio. In some experiments, trypsin was then added at a 1:100 protease-to-protein ratio followed by overnight incubation at 37° C. The reaction was quenched with 1% formic acid, subjected to C18 solid-phase extraction (Sep-Pak, Waters) and vacuum-centrifuged to near-dryness.

Peptide Labeling with Tandem Mass Tags (TMT)

In preparation for TMT labeling, desalted peptides were dissolved in 100 mM HEPES pH 8.5. For whole proteome samples, peptide concentration was measured by microBCA (Pierce), and 100 μg of peptide labeled with TMT reagent. For plasma membrane samples, 100% of each peptide sample was labeled.

TMT reagents (0.8 mg) were dissolved in 40 μL anhydrous acetonitrile and 10 μL (whole proteome) or 2.5 μl (PM samples) added to peptide at a final acetonitrile concentration of 30% (v/v). For experiments PM1 and WCL1 (described below), samples were labeled as follows: mock replicate 1 (TMT 126); mock replicate 2 (TMT 128); 24 hour infection replicate 1 (TMT 127n); 24 hour infection replicate 2 (TMT 127c); 48 hour infection replicate 1 (TMT 129n); 48 h infection replicate 2 (TMT 129c); 72 h infection replicate 1 (TMT 130); 72 hour infection replicate 2 (TMT 131). Following incubation at room temperature for 1 hour, the reaction was quenched with hydroxylamine to a final concentration of 0.3% (v/v). TMT-labeled samples were combined at a 1:1:1:1:1:1:1:1 ratio (8-plex TMT) or 1:1:1:1:1:1:1:1:1:1 ratio (10-plex TMT). The sample was vacuum-centrifuged to near dryness and subjected to C18 solid-phase extraction (SPE) (Sep-Pak, Waters).

### Offline High pH Reversed-Phase (HPRP) Fractionation

TMT-labeled peptide samples were fractionated using an Agilent 300Extend C18 column (5 μm particles, 4.6 mm ID, 220 mm length) and an Agilent 1100 quaternary pump equipped with a degasser and a photodiode array detector (220 and 280 nm, ThermoFisher, Waltham, Mass.). Peptides were separated with a gradient of 5% to 35% acetonitrile in 10 mM ammonium bicarbonate pH 8 over 60 min. 96 resulting fractions were consolidated into 12, acidified to 1% formic acid and vacuum-centrifuged to near dryness. Each fraction was desalted using a StageTip, dried, and reconstituted in 4% acetonitrile/5% formic acid prior to LC-MS/MS.

### Offline Tip-Based Strong Cation Exchange (SCX) Fractionation

The protocol for solid-phase extraction based SCX peptide fractionation described in Dephoure and Gygi, Methods 54:379-386 (2011), incorporated by reference in its entirety, was modified for small peptide amounts. Briefly, 10 mg of PolySulfoethyl A bulk material (Nest Group Inc) was loaded into a fitted 200 ul tip in 100% Methanol using a vacuum manifold. SCX material was conditioned slowly with 1 ml SCX buffer A (7 mM KH₂PO₄, pH 2.65, 30% Acetonitrile), then 0.5 ml SCX buffer B (7 mM KH₂PO₄, pH 2.65, 350 mM KCl, 30% Acetonitrile) then 2 ml SCX buffer A. Dried peptides were resuspended in 500 μl SCX buffer A and added to the tip at a flow rate of ˜150 μl/min, followed by a 150 μl wash with SCX buffer A. Fractions were eluted in 150 ul buffer at increasing K⁺ concentrations (10, 24, 40, 60, 90, 150 mM KCl), vacuum-centrifuged to near dryness then desalted using Stage Tips.

### Liquid Chromatography and Tandem Mass Spectrometry

Mass spectrometry data was acquired using an Orbitrap Elite mass spectrometer (Thermo Fisher Scientific, San Jose, Calif.) coupled with a Proxeon EASY-nLC II liquid chromatography (LC) pump (Thermo Fisher Scientific). Peptides were separated on a 100 μm inner diameter microcapillary column packed with 0.5 cm of Magic C4 resin (5 μm, 100 Å, Michrom Bioresources) followed by ˜20 cm of Maccel C18 resin (3 μm, 200 Å, Nest Group).

Peptides were separated using a 3 hour gradient of 6% to 30% acetonitrile in 0.125% formic acid at a flow rate of 300 nL/min. Each analysis used an MS3-based TMT method. The scan sequence began with an MS1 spectrum (Orbitrap analysis, resolution 60,000, 300-1500 Th, AGC target 1×10⁶, maximum injection time 150 ms). The top ten precursors were then selected for MS2/MS3 analysis. MS2 analysis consisted of CID (quadrupole ion trap analysis, AGC 2×10³, NCE 35, q-value 0.25, maximum injection time 100 ms). Following acquisition of each MS2 spectrum, we collected an MS3 spectrum using our recently described method in which multiple MS2 fragment ions are captured in the MS3 precursor population using isolation waveforms with multiple frequency notches. MS3 precursors were fragmented by HCD and analyzed using the Orbitrap (NCE 50, max AGC 1.5×10⁵, maximum injection time 250 ms, isolation specificity 0.8 Th, resolution was 30,000 at 400 Th).

### Data Analysis

Mass spectra were processed using a Sequest-based software pipeline. MS spectra were converted to mzXML using a modified version of ReAdW.exe. A combined database was constructed from (a) the human Uniprot database (Aug. 10, 2011), (b) the human cytomegalovirus (strain Merlin) Uniprot database, (c) all additional novel human cytomegalovirus ORFs described in Stern-Ginossar et al., Science 338:1088-1093 (2012), hereby incorporated by reference, and (d) common contaminants such as porcine trypsin and endoproteinase LysC. The combined database was concatenated with a reverse database composed of all protein sequences in reversed order. Searches were performed using a 20 ppm precursor ion tolerance. Product ion tolerance was set to 0.03 Th. TMT tags on lysine residues and peptide N termini (229.162932 Da) and carbamidomethylation of cysteine residues (57.02146 Da) were set as static modifications, while oxidation of methionine residues (15.99492 Da) was set as a variable modification.

Peptide spectral matches (PSMs) were filtered to an initial peptide-level FDR of 1% with subsequent filtering to attain a final protein-level FDR of 1%. PSM filtering was performed using a linear discriminant analysis, considering the following parameters: XCorr, ΔCn, missed cleavages, peptide length, charge state, and precursor mass accuracy. Protein assembly was guided by principles of parsimony to produce the smallest set of proteins necessary to account for all observed peptides. Where all PSMs from a given HCMV protein could be explained either by a canonical gene or novel ORF, the canonical gene was picked in preference.

For TMT-based reporter ion quantitation, we extracted the signal-to-noise (S/N) ratio for each TMT channel and found the closest matching centroid to the expected mass of the TMT reporter ion. Proteins were quantified by summing reporter ion counts across all matching peptide-spectral matches using in-house software. Briefly, a 0.003 Th window around the theoretical m/z of each reporter ion (126, 127n, 127c, 128n, 128c, 129n, 129c, 130n, 130c, 131) was scanned for ions, and the maximum intensity nearest to the theoretical m/z was used. Peptide-spectral matches with poor quality MS3 spectra (more than 9 TMT channels missing and/or a combined S/N of less than 100 across all TMT reporter ions) or no MS3 spectra at all were excluded from quantitation. All MS2 and MS3 spectra from novel ORFs were all manually validated to confirm both identifications and quantifications. Protein quantitation values were exported for further analysis in Excel.

For protein quantitation, reverse and contaminant proteins were removed, then each reporter ion channel was summed across all quantified proteins and normalized assuming equal protein loading across all 8 or 10 samples. Gene Ontology and KEGG terms were added using Perseus version 1.4.1.3. Gene name aliases were added using GeneALaCart (www.genecards.org). The one-way ANOVA test was used to identify proteins differentially expressed over time in experiments PM1 and WCL1, and was corrected using the method of Benjamini-Hochberg to control for multiple testing error (Benjamini and Hochberg, J. R. Stat. Soc. Ser. B-Methodol. 57:289-300 (1995), hereby incorporated by reference. A Benjamini-Hochberg-corrected p-value <0.05 was considered statistically significant. Values were calculated using Mathematica (Wolfram Research). Other statistical analyses including Principal Component analysis and k-means clustering were performed using XLStat (Addinsoft). Hierarchical centroid clustering based on uncentered Pearson correlation was performed using Cluster 3.0 (Stanford University) and visualized using Java Treeview (http://jtreeview.sourceforge.net) unless otherwise noted. For RNAseq data from Stern-Ginnosar et al, mRNA reads densities from 5, 24 and 72 h for each transcript were normalized to 1, and hierarchical clustering based on Euclidian distance was performed using Cluster 3.0.

### Example 1

### Validation of Quantitative Temporal Viromics (QTV)

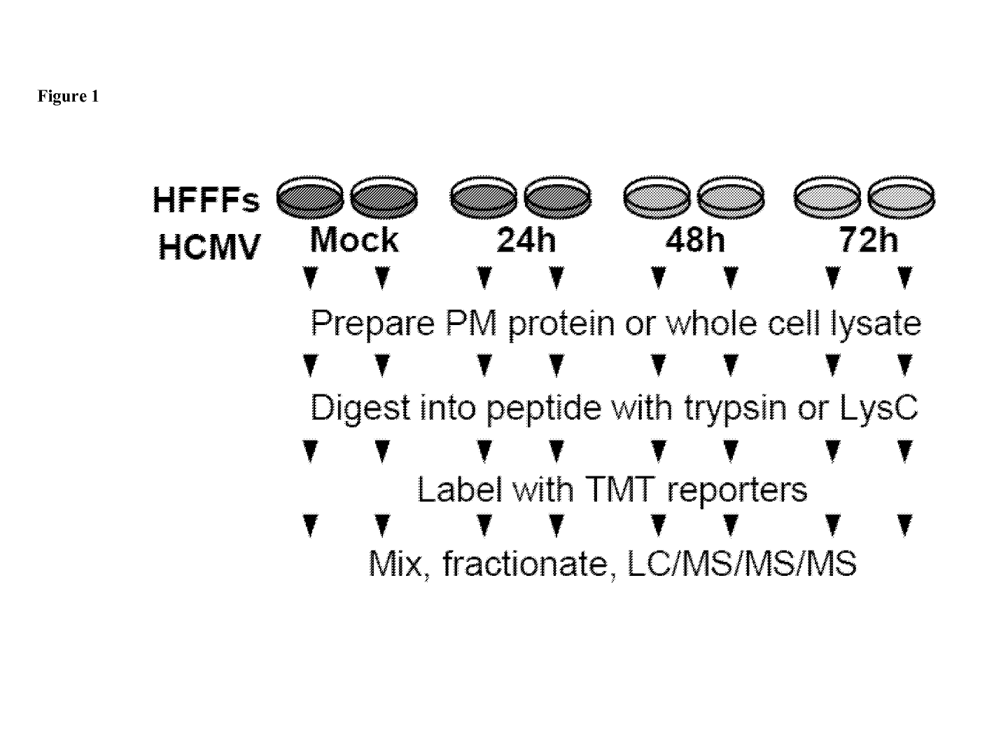

Primary human fetal foreskin fibroblasts (HFFF) were infected with the clinical HCMV strain Merlin as described above and plasma membrane profiling (PMP) was used to measure changes in plasma membrane receptor expression. Initially, 8-plex TMT were used to assess in biological duplicate three of the key time points in productive HCMV infection and mock infection (experiment PM1, FIG. 1). In total, 927 PM proteins were quantified. Among the proteins quantified, the cell surface expression level of 56% of the proteins changed by more than 2 fold, and 33% by more than 3-fold at 72 hours after infection. Replicate experiments clustered tightly.

HCMV protein UL138 degrades the cell surface ABC transporter Multidrug Resistance-associated Protein-1 (ABCC1) in both productive and latent infection, and ABCC1-specific cytotoxic substrate Vincristine can be used therapeutically to eliminate cells latently infected with HCMV (Weekes et al., Science 340:199-202 (2013), hereby incorporated by reference in its entirety).

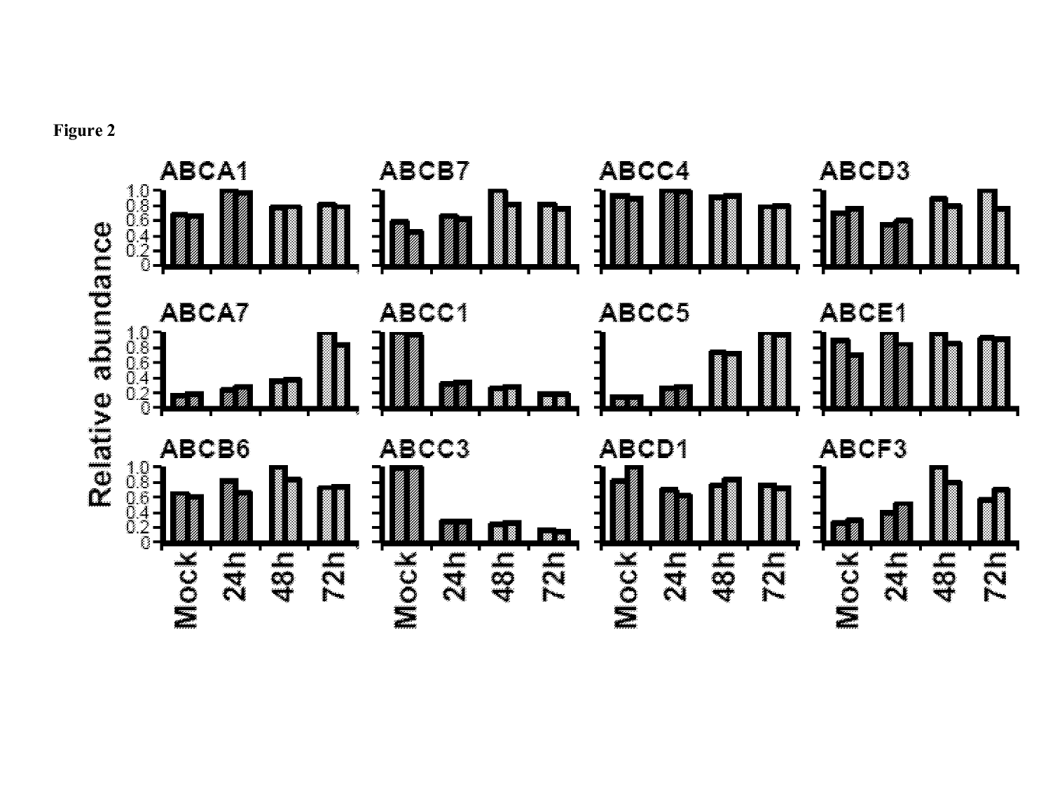

To validate the PMP procedure, all quantified ABC transporters were examined, and selective ABCC1 downregulation was confirmed (FIG. 2). Multidrug Resistance-associated Protein 3 (ABCC3) was downregulated with very similar kinetics, indicating that this drug transporter represents an additional therapeutic target. To identify additional therapeutic targets, changes the cell surface expression of other transporters were also examined. As with ABCC1 and ABCC3, sodium-coupled neutral amino acid transporter 4 (SLC38A4) and solute carrier family 2, facilitated glucose transporter member 10 (SLC2A10) were also downregulated, providing additional therapeutic targets.

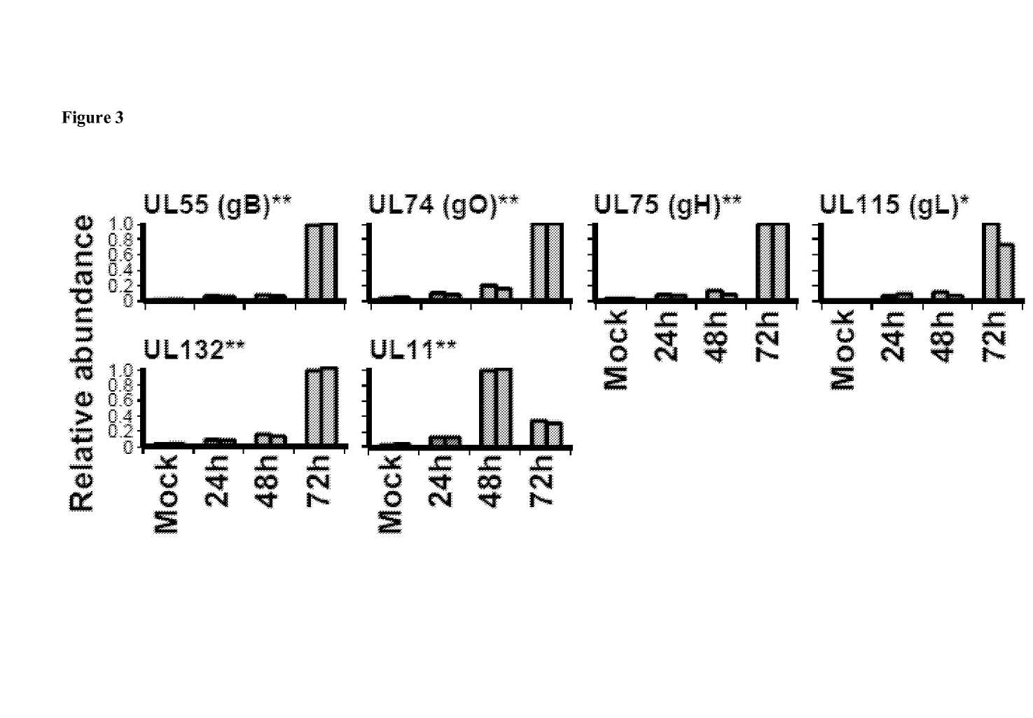

The instant methodology was further validated by the detection of the upregulation of all six HCMV proteins previously reported as being present at the plasma membrane of HCMV infected cells (FIG. 3).

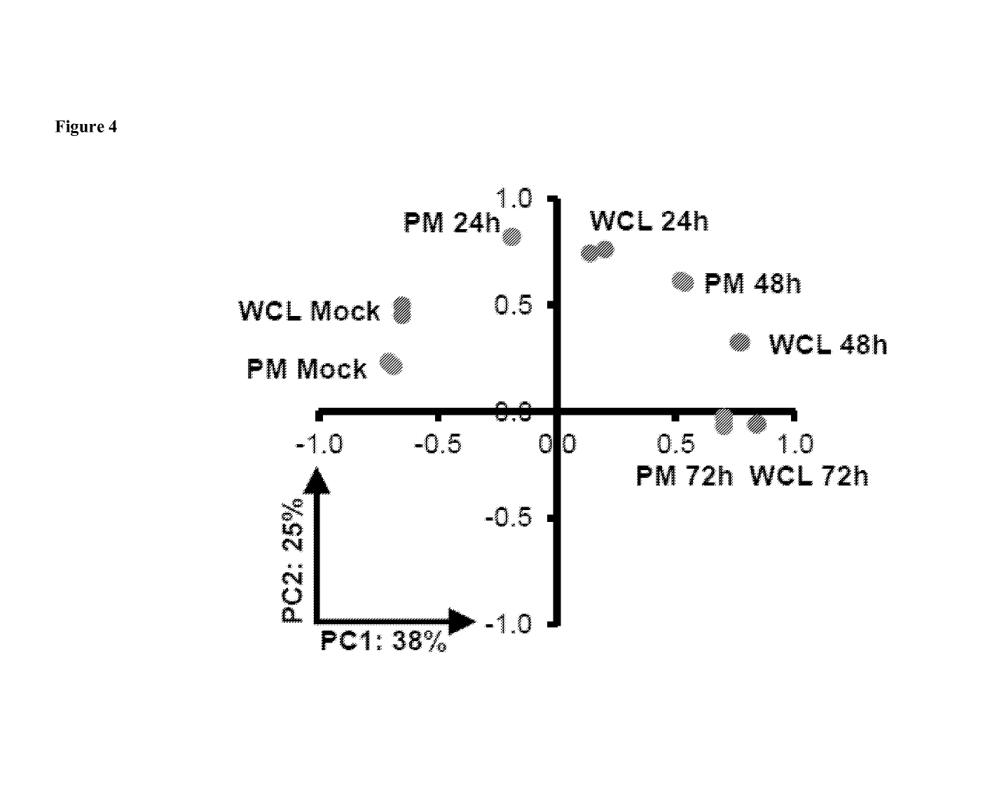

Temporal analysis of whole cell lysates (WCLs) of HCMV-infected HFFFs enables the study of changes in expression of intracellular proteins during infection and a comparison of the total abundance of a given protein to its expression at the plasma membrane. Analyzing HFFF infected with PMP samples revealed a high degree of reproducibility amongst biological replicates (WCL1, FIG. 4).

### Example 2

### Cell Surface Receptors Modulated by HCMV

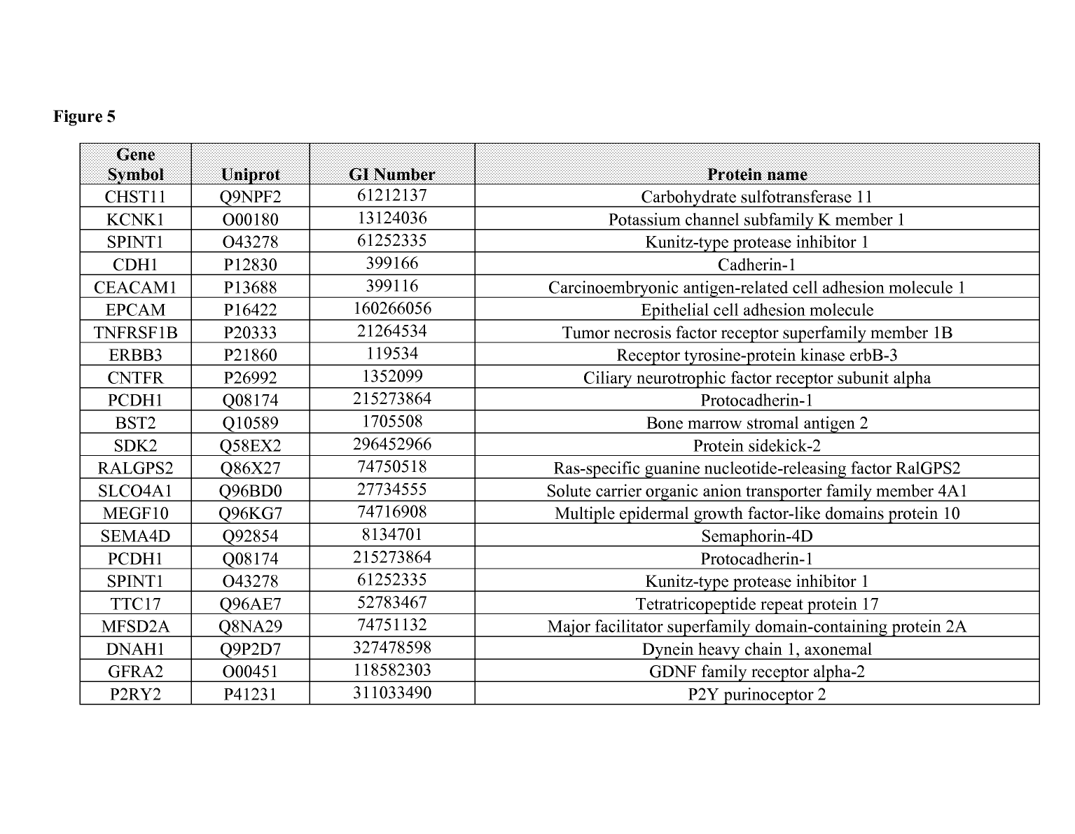

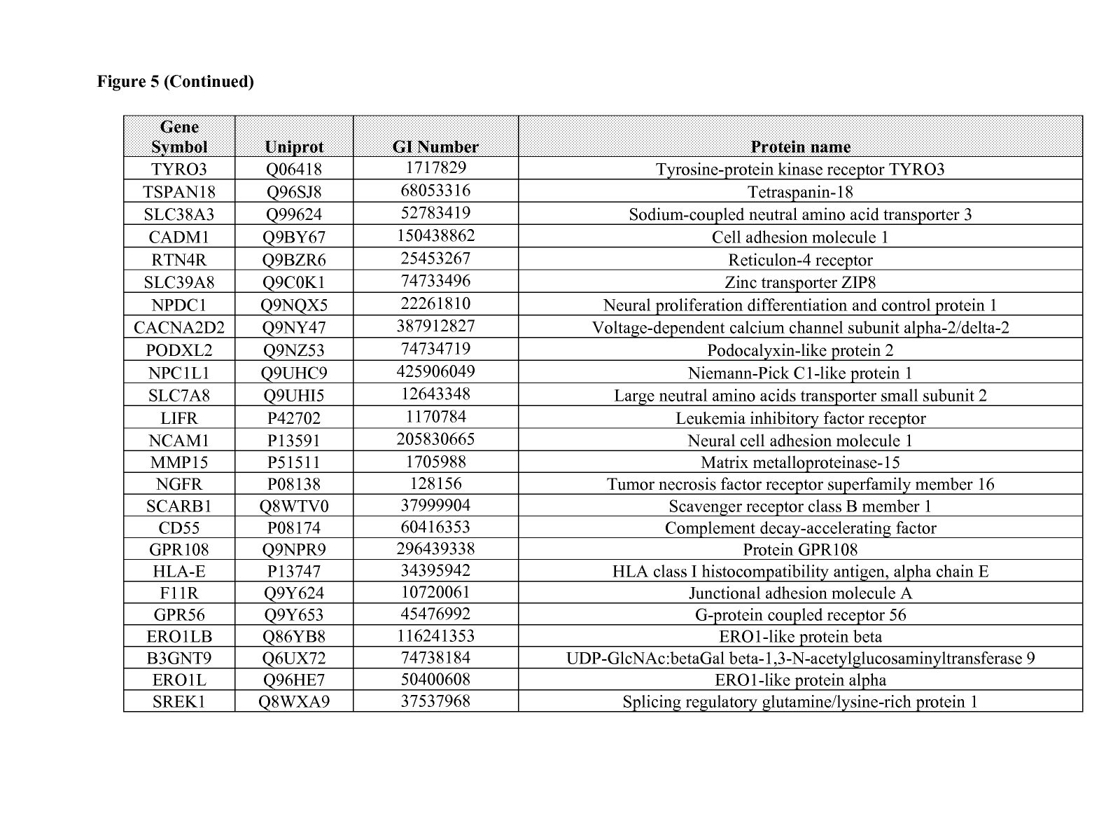

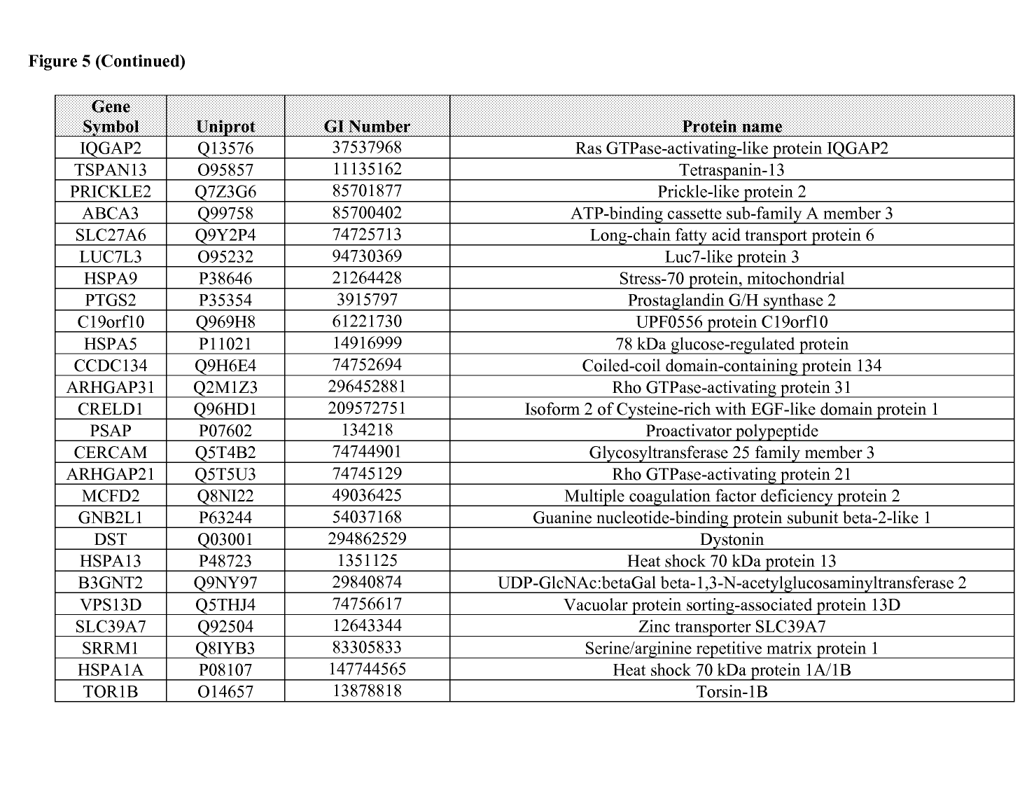

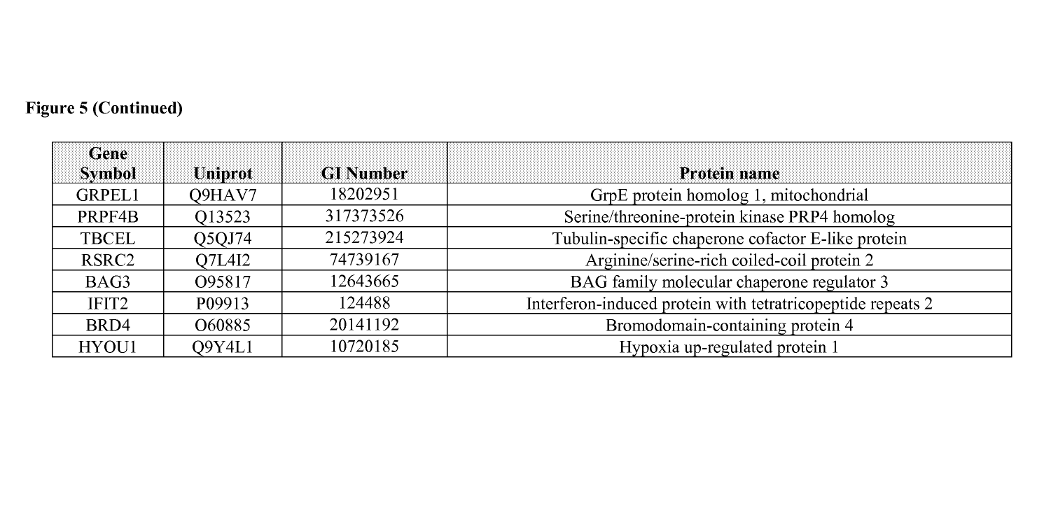

The QTV procedure described above was used to follow the cell surface expression of endogenous proteins following HCMV infection. Data generated using the QTV procedure was analyzed to identify cell-surface proteins that were rapidly upregulated on the surface of HCMV infected cells but not on the surface of mock-infected cells (FIG. 5). Due to their early and selective expression on HCMV infected cells, the proteins listed in FIG. 5 can be used to selectively identify HCMV infected cells soon after viral infection and are attractive targets for novel HCMV therapeutics.

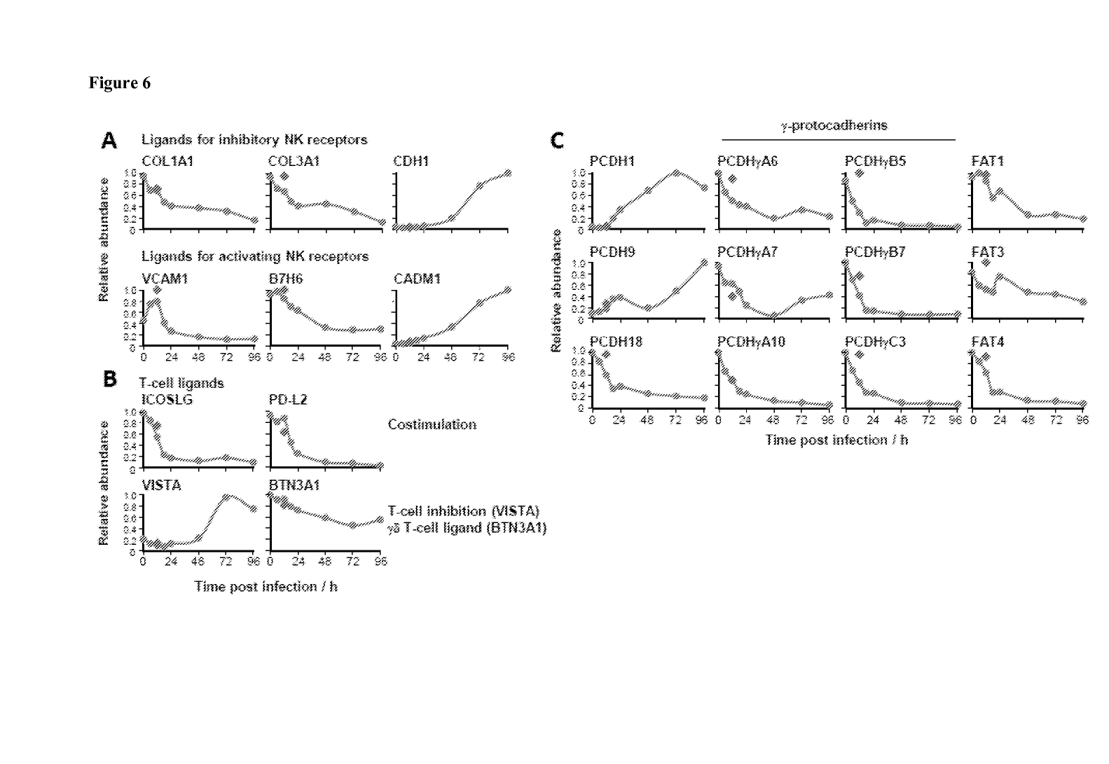

A number of NK cell ligands were identified as having altered plasma membrane expression following HCMV infection (FIG. 6). For example, E-cadherin (CDH1), the ligand for the inhibitory NK receptor KLRG-1 (killer cell lectin-like receptor subfamily G member 1) was dramatically upregulated during infection (FIG. 6A). Vascular cell adhesion molecule 1 (VCAM1) and B7H6, ligands for activating NK receptors a4131 integrin and NKp30 were downregulated during viral infection (FIG. 6A).

A similar screen was performed for all known αβ T-cell costimulatory molecules, and γδ T-cell ligands. The T-cell costimulators ICOSLG (inducible T-cell co-stimulator ligand) and PD-L2 (PDCD1LG2) were downregulated during infection, as was butyrophilin subfamily 3 member A1 (BTN3A1), which is recognized by Vγ9Vδ2+ T-cells. V-domain Ig suppressor of T cell activation (VISTA, C10Orf54), a novel B7 family inhibitory ligand was upregulated late in infection (FIG. 6B).

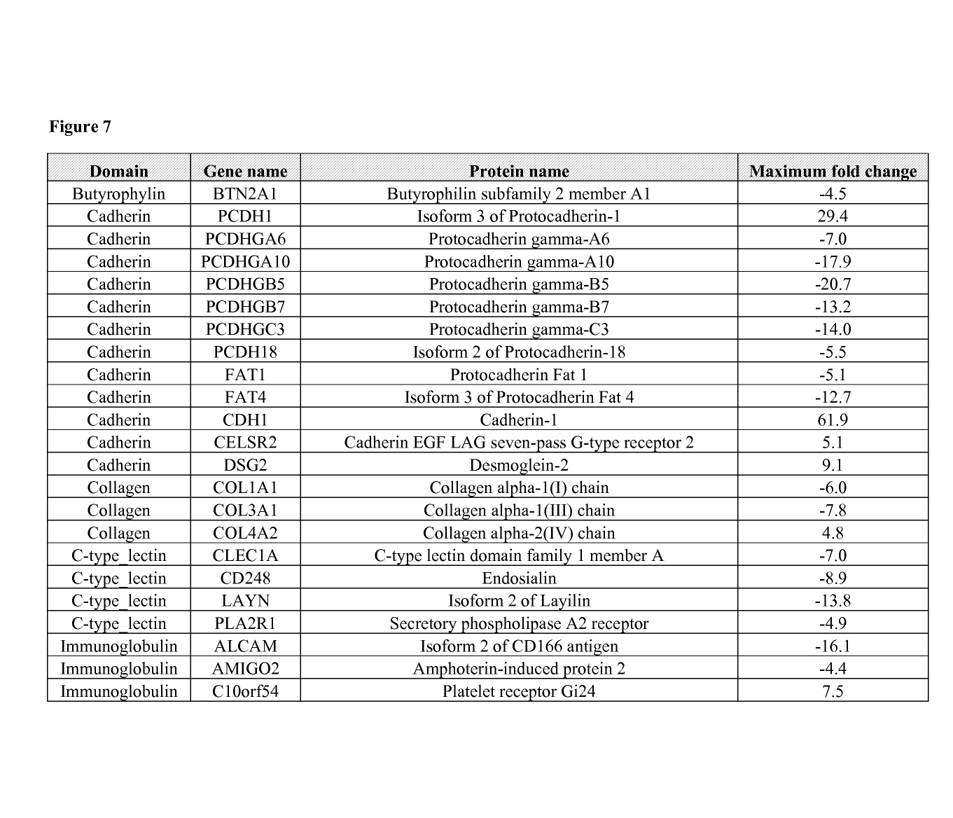

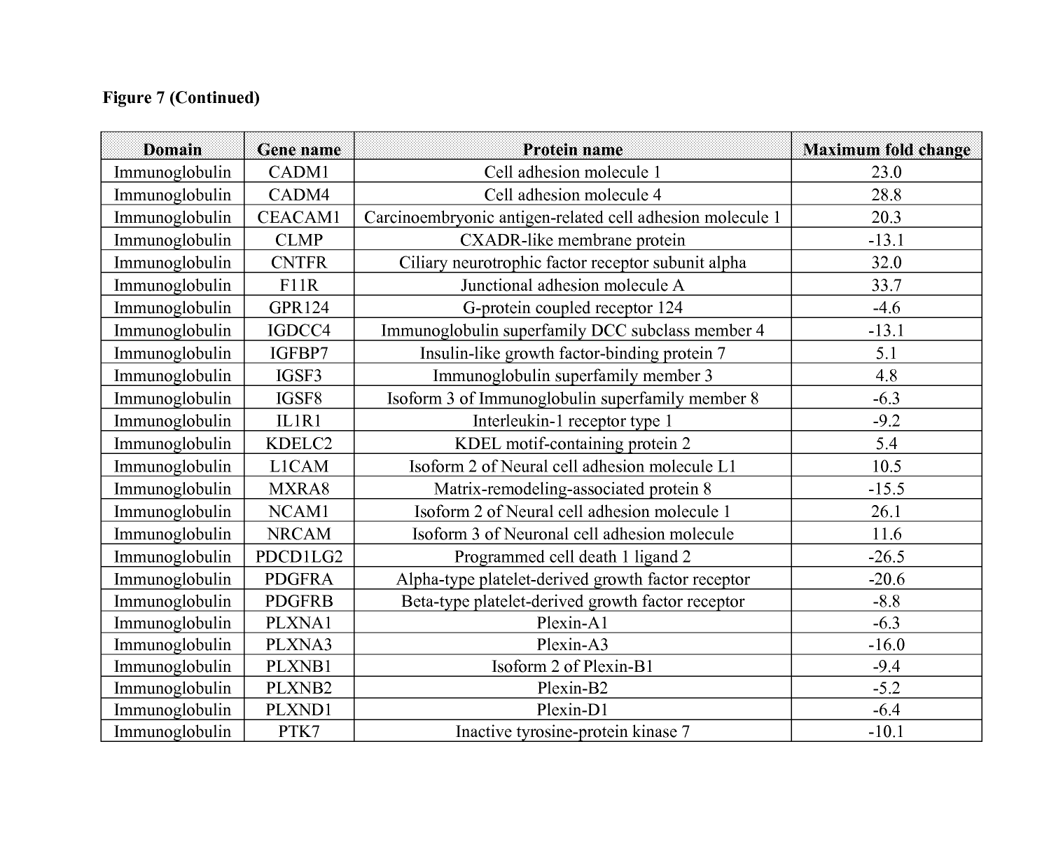

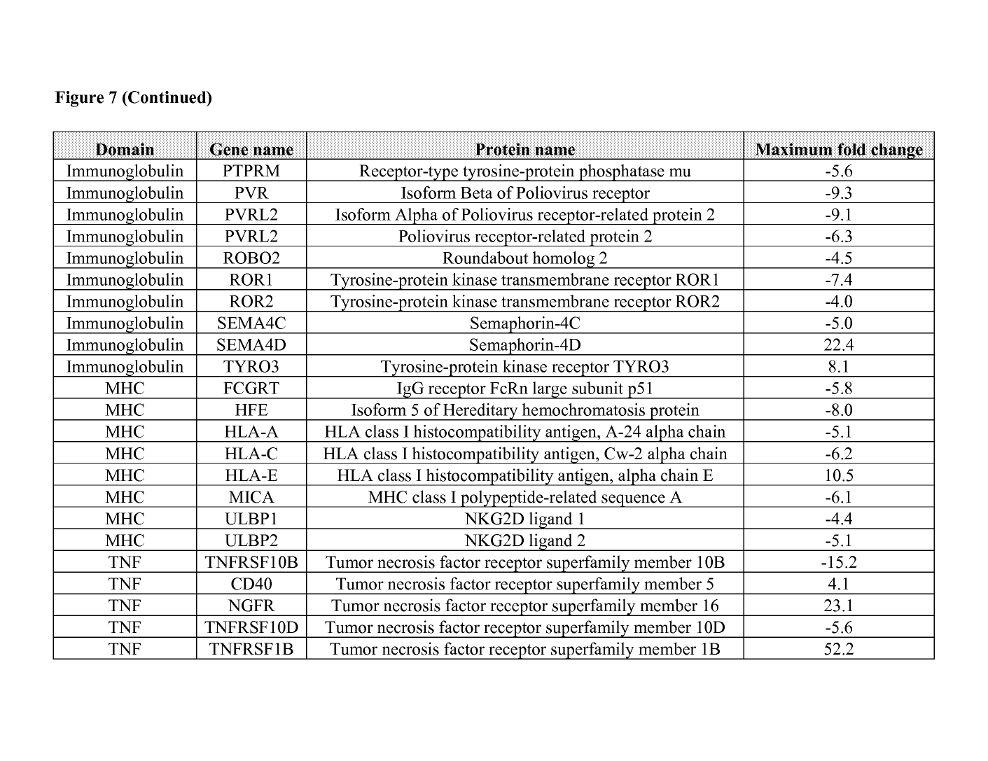

Known NK and T-cell ligands generally belong to a small number of protein families, including Cadherins, C-type lectins, Immunoglobulin, TNF and major histocompatibility complex (MHC)-related molecules. To discover novel ligands, InterPro functional domain annotations were added to data from experiments PM1 and PM2. Analysis of the resulting data identified 74 proteins that had relevant InterPro annotation and at least a 4-fold change in cell surface expression following infection (FIG. 7). Eight downregulated proteins were protocadherins, and all six quantified γ-protocadherins were potently downregulated (FIG. 6C). The protocadherins therefore represent a major class of immunoreceptors.

There is increasing evidence for a substantial role of plexin-semaphorin signaling in the immune system. For example, secreted class III semapohrins bind plexins A and D1 to regulate migration of dendritic cells to secondary lymphoid organs. Plexin B2 interacts with membrane-bound semaphorin 4D to promote epidermal γδ T-cell activation. HCMV substantially downregulated five of the nine plexins, A1, A3, B1, B2 and D1. Neuropilin 2, a plexin co-receptor was also rapidly downregulated. Semaphorin 4D was dramatically upregulated and 4C downregulated (FIG. 7).

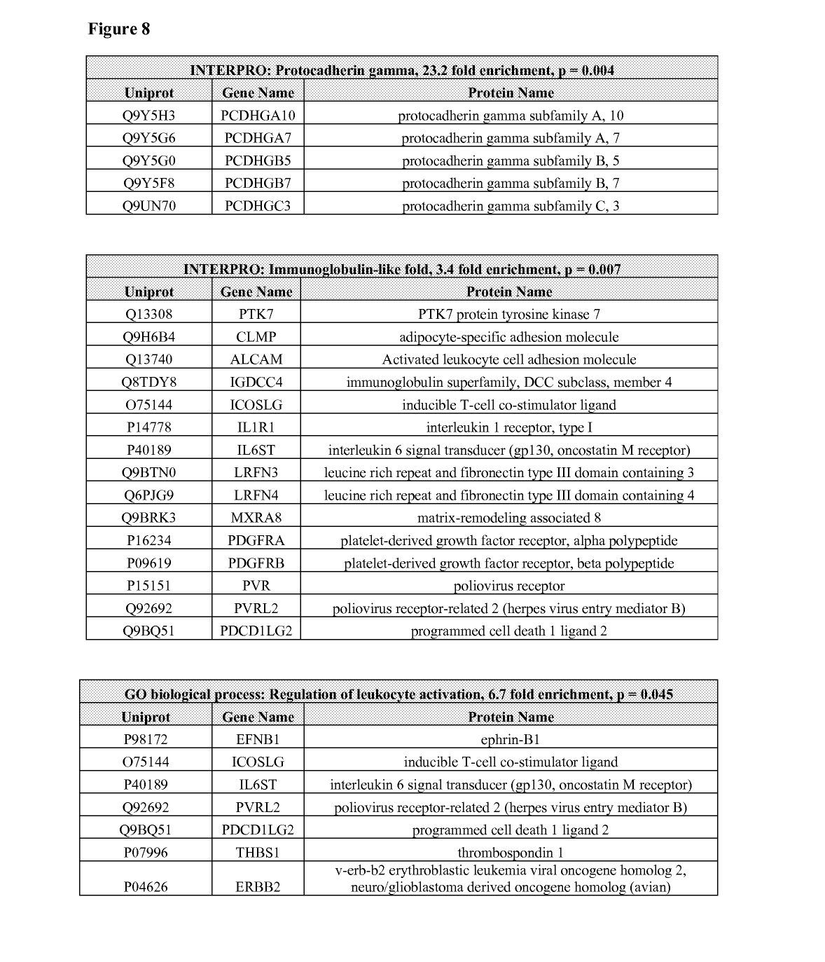

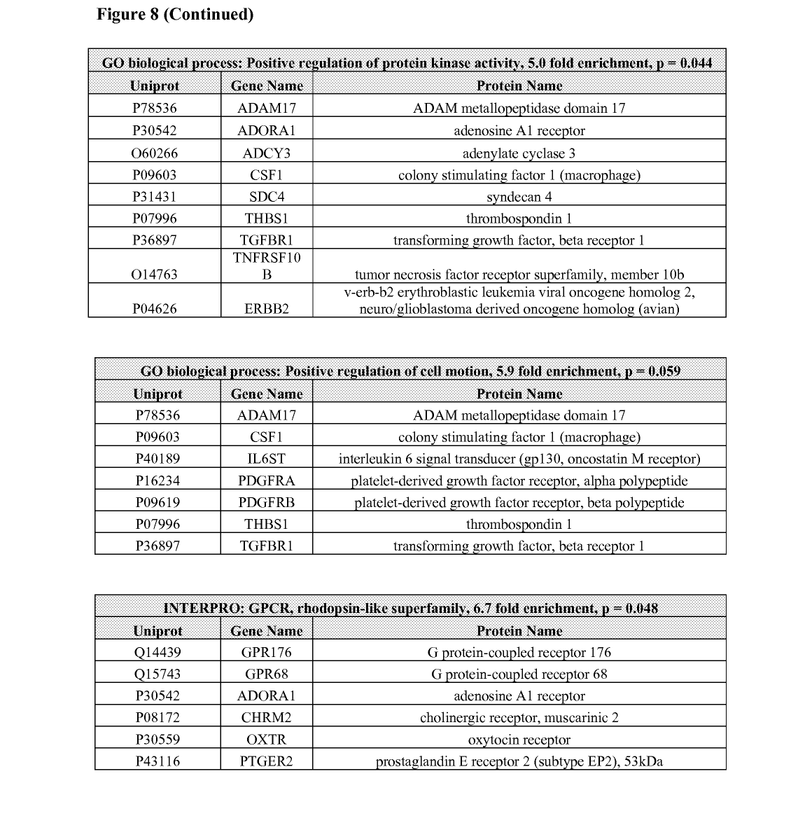

DAVID software was used to determine which functional protein categories were enriched within highly downregulated PM proteins. The Interpro categories ‘protocadherin gamma’ and ‘immunoglobulin-like fold’ were significantly enriched in addition to Gene Ontology (GO) biological processes ‘regulation of leukocyte activation’ and ‘positive regulation of cell motion’. DAVID analysis also revealed novel families of downregulated proteins, including six rhodopsin-like superfamily G-protein coupled receptors (FIG. 8).

### Example 3

### Temporal Analysis of HCMV Viral Protein Expression

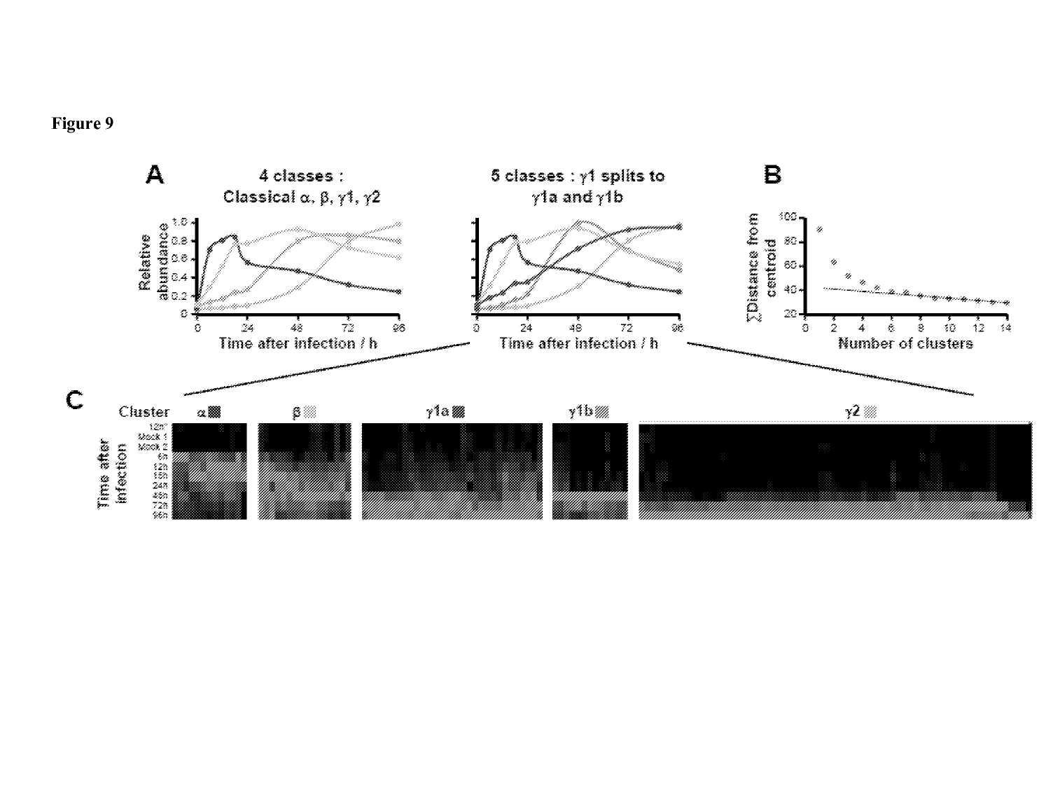

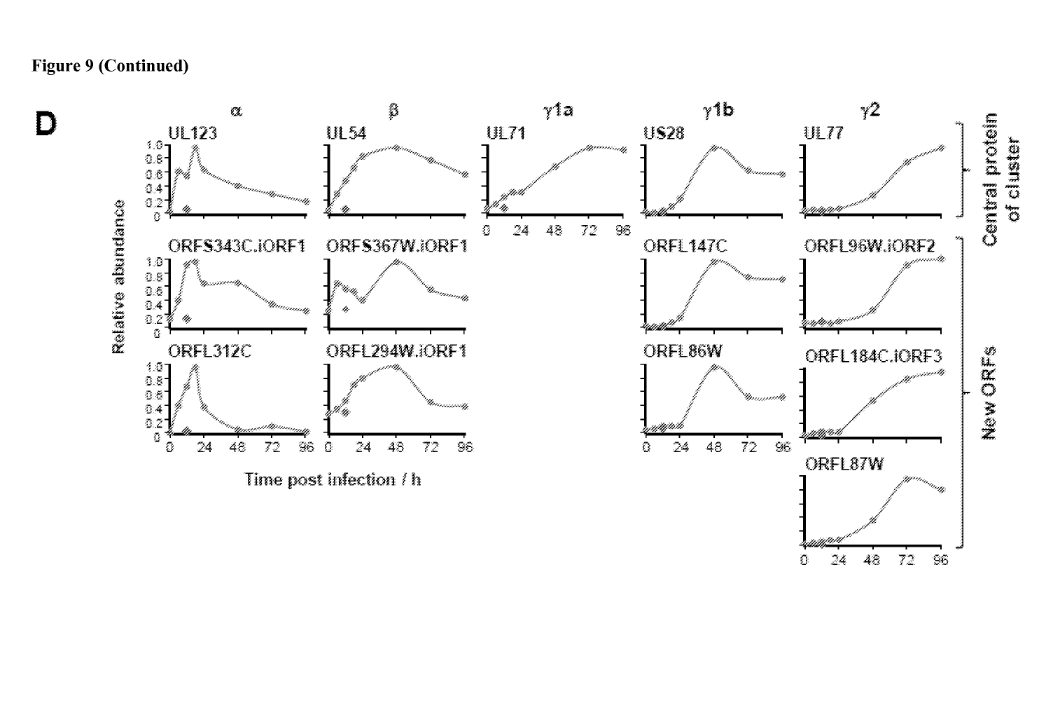

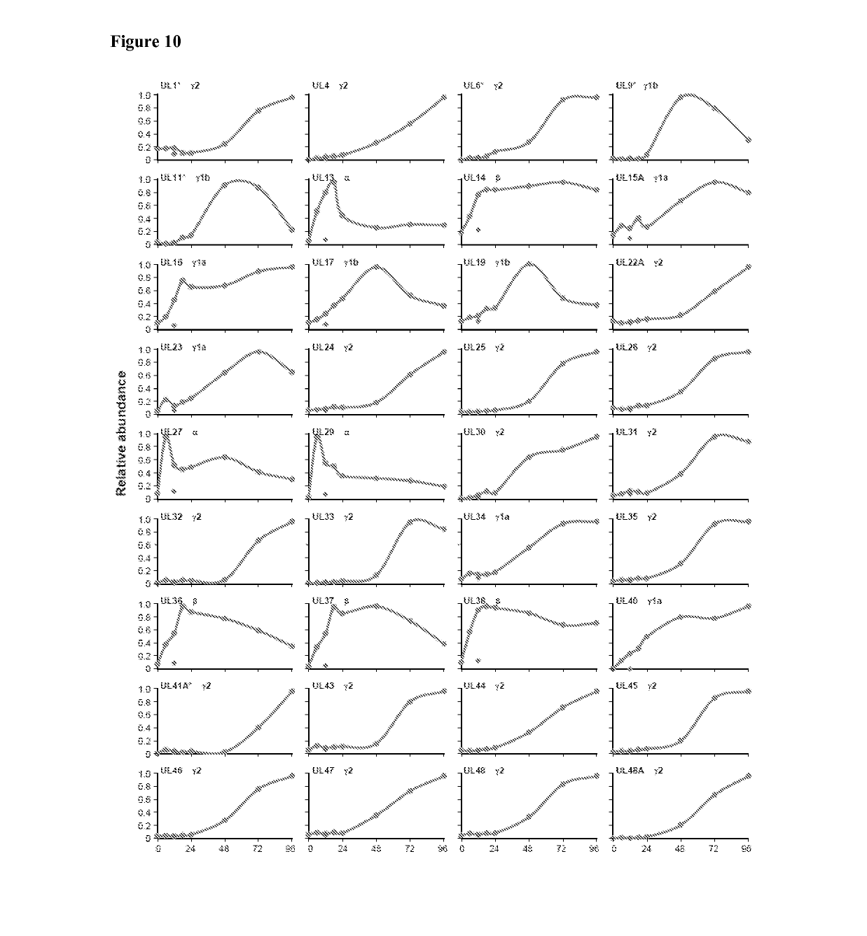

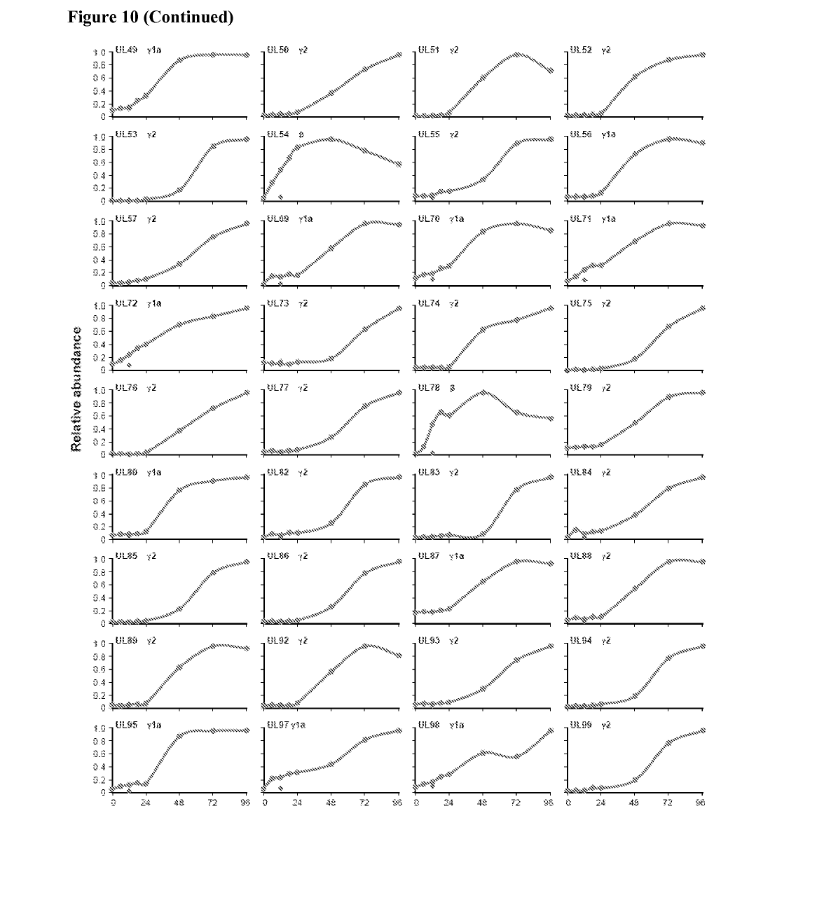

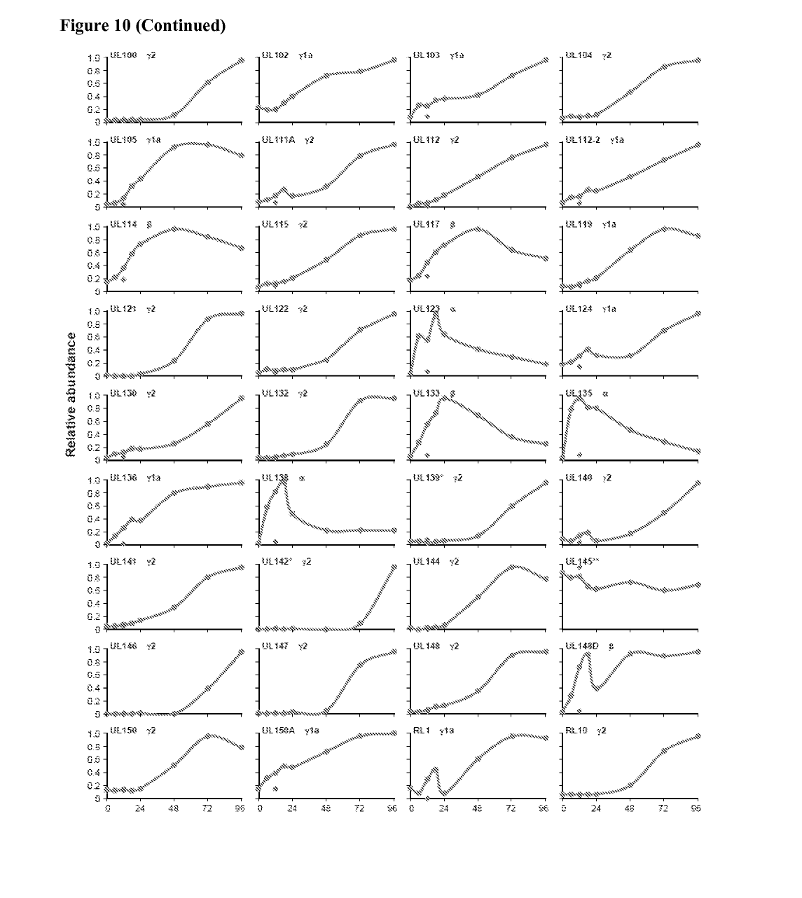

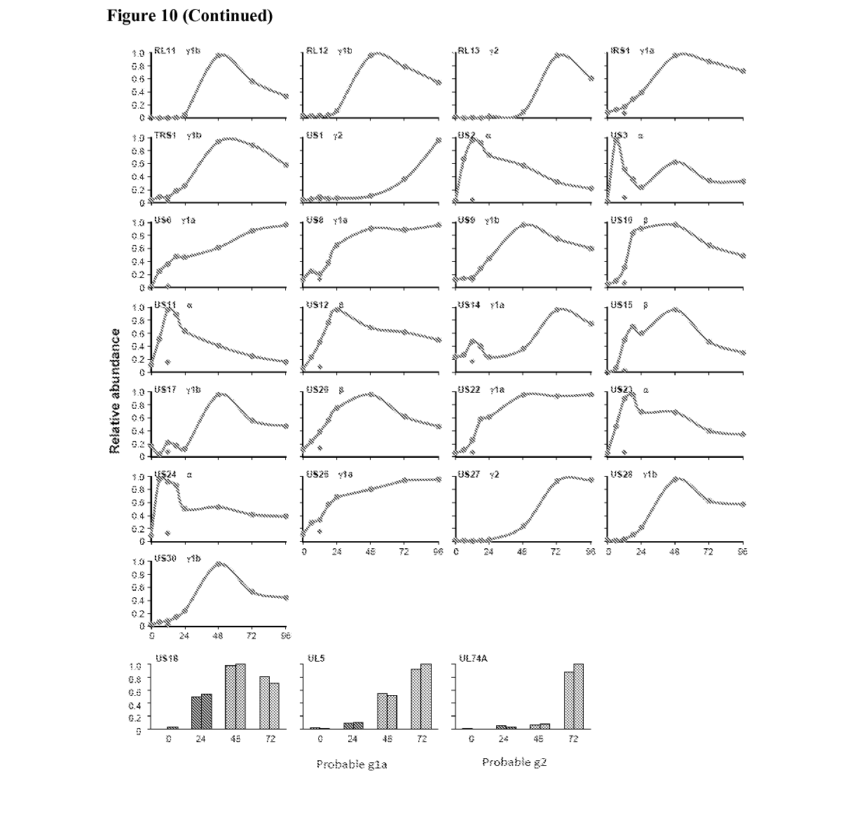

Using the methods described herein above, the changes in the expression of the majority (136/171) of canonical HCMV proteins and 9 novel ORFs was quantified in one experiment (FIGS. 9, 10).

The k-means method is useful to cluster viral proteins into classes based on the similarity of temporal profiles, and it is possible to specify the number of classes to be considered. With 4 classes, proteins grouped according to the temporal cascade of α, β, γ1, γ2 (FIG. 9A). To determine how many true classes of HCMV genes actually exist, k-means clustering was performed with 2-14 classes and the summed distance of each protein from its cluster centroid was assessed. The point of inflexion fell between 5-7 classes, suggesting that there are at least 5 distinct profiles of viral protein expression (FIG. 9B).

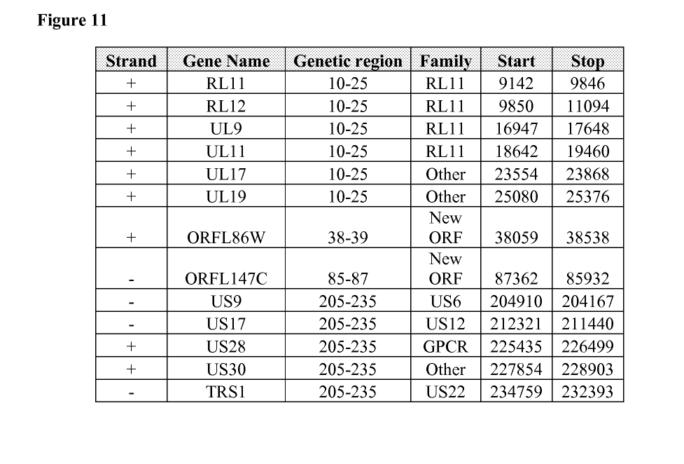

A cluster of 13 early-late proteins referred to herein as γ1b exhibited a distinct profile to other γ1a early-late proteins, (FIGS. 9C-D), with maximal expression at 48 h and low expression at other time points. Members of this cluster predominantly originated from two regions of the viral genome, and four belonged to the RL11 family (FIG. 11).

Eight HCMV proteins are expressed earlier in infection than had previously been supposed. UL27, UL29, UL135, UL138, US2, US11, US23 and US24 all exhibited peak expression at between 6-18 hours post infection. UL29 and US24 appeared particularly early, with peak expression at only 6 hours post infection.

The immediate early gene IE2 (UL122, γ2) demonstrated very little protein expression prior to 48 h. UL122 and UL123 are encoded by alternative splicing of a single major immediate-early transcript. Exons 1, 2, 3 and 4 encode UL123 and exons 1, 2, 3 and 5 encode UL122 and additional transcripts have also been detected from the region of exon 5. Each peptide quantified from every exon was identified (FIG. 10). The expression of all peptides from exon 4 peaked at 18-24 h, corresponding to the predicted expression of UL123 protein. Ten exon 5 peptides corresponding to the internal ORF, ORFL265C.iORF1 were maximally expressed at 96 h, whereas a single peptide N-terminal to this ORF had a distinct profile with earlier expression. This indicates the existence of at least two proteins arising wholly or in part from exon 5, and corresponds to the known late expression of ORFL265C.iORF1 transcript.

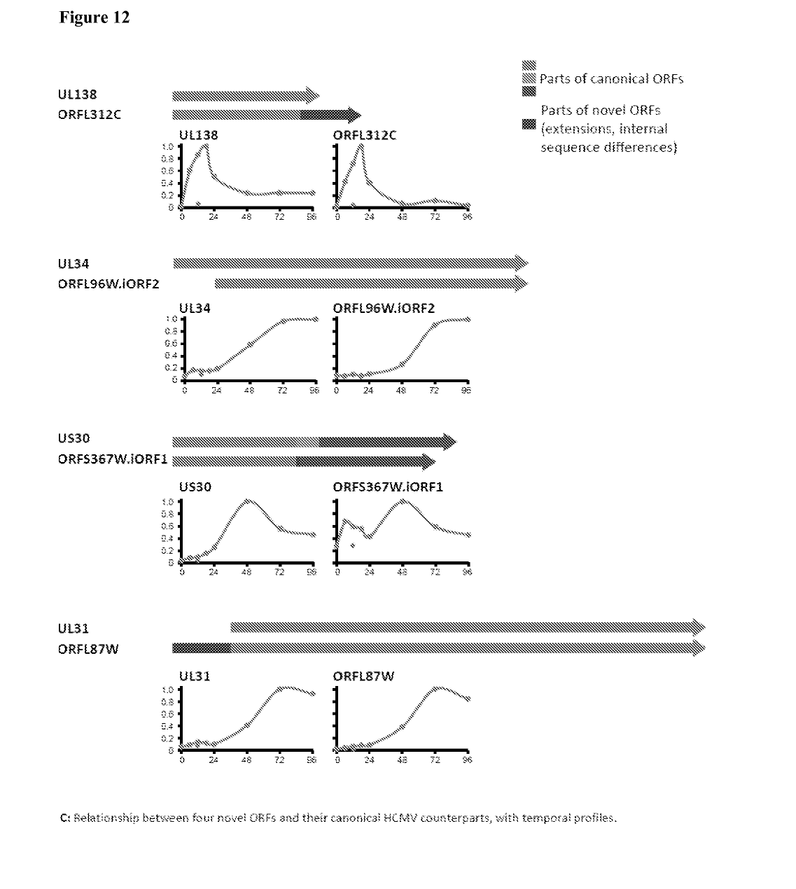

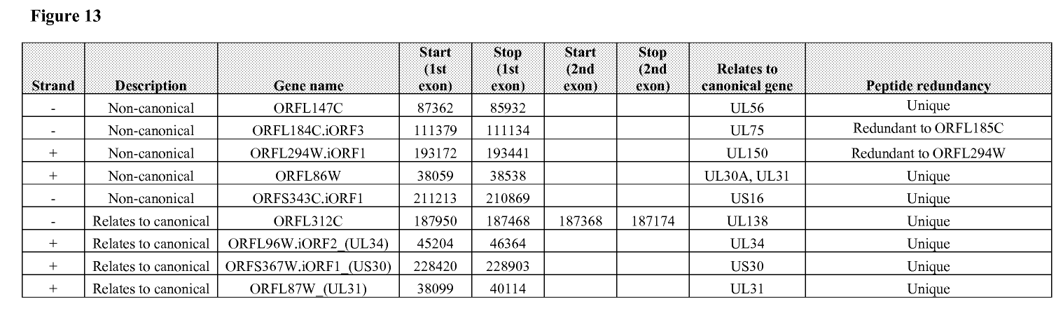

Nine novel ORFs belonging to α, β, γ1b or γ2 classes were identified (FIG. 9C). Four ORFs related to canonical HCMV proteins (N-terminal extension, internal ORF, C-terminal extension) and demonstrated similar temporal profiles to their canonical counterparts (FIG. 12). Five ORFs were non-canonical, encoded either in different reading frames, or on the opposite strand to canonical genes (FIG. 13).

### Example 4

### HCMV Proteins Present at the Cell Surface

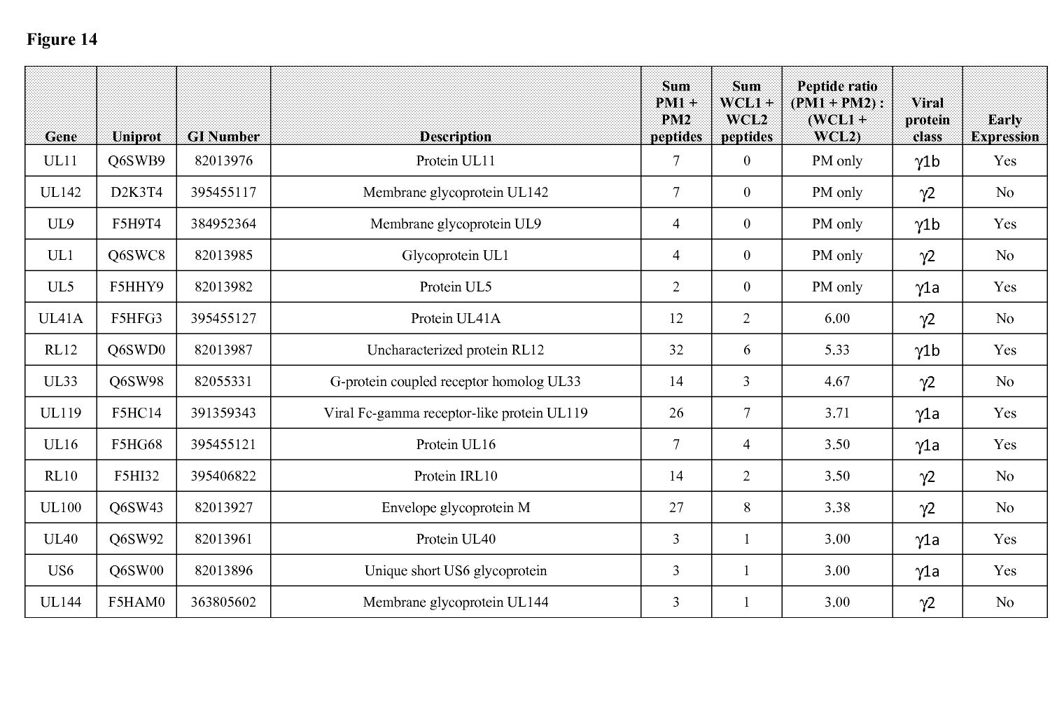

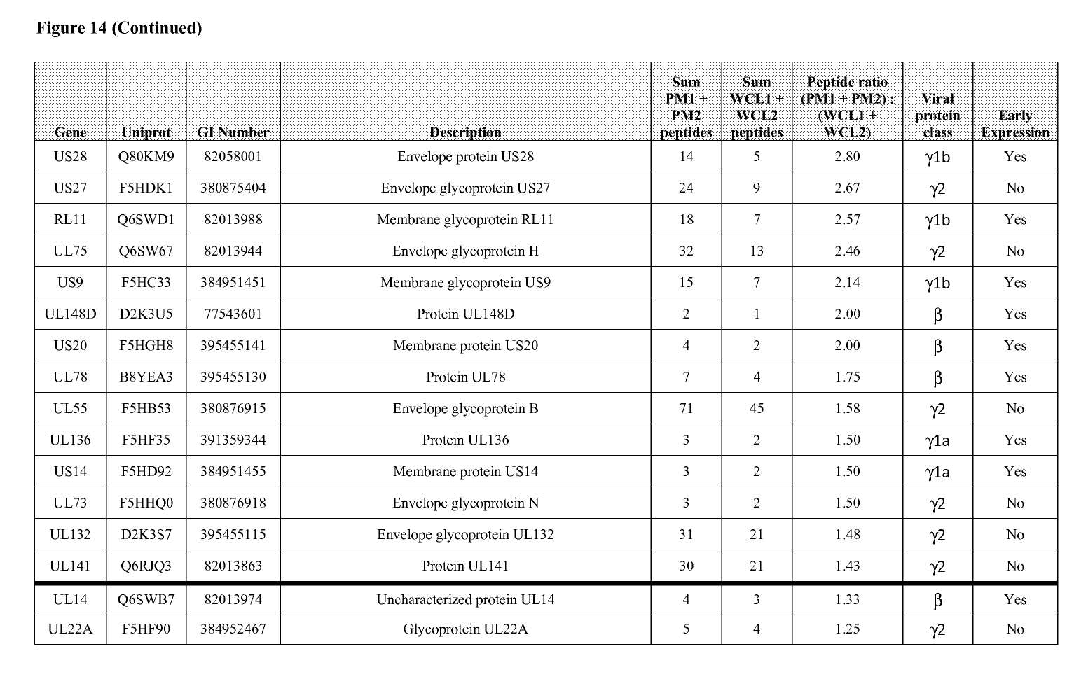

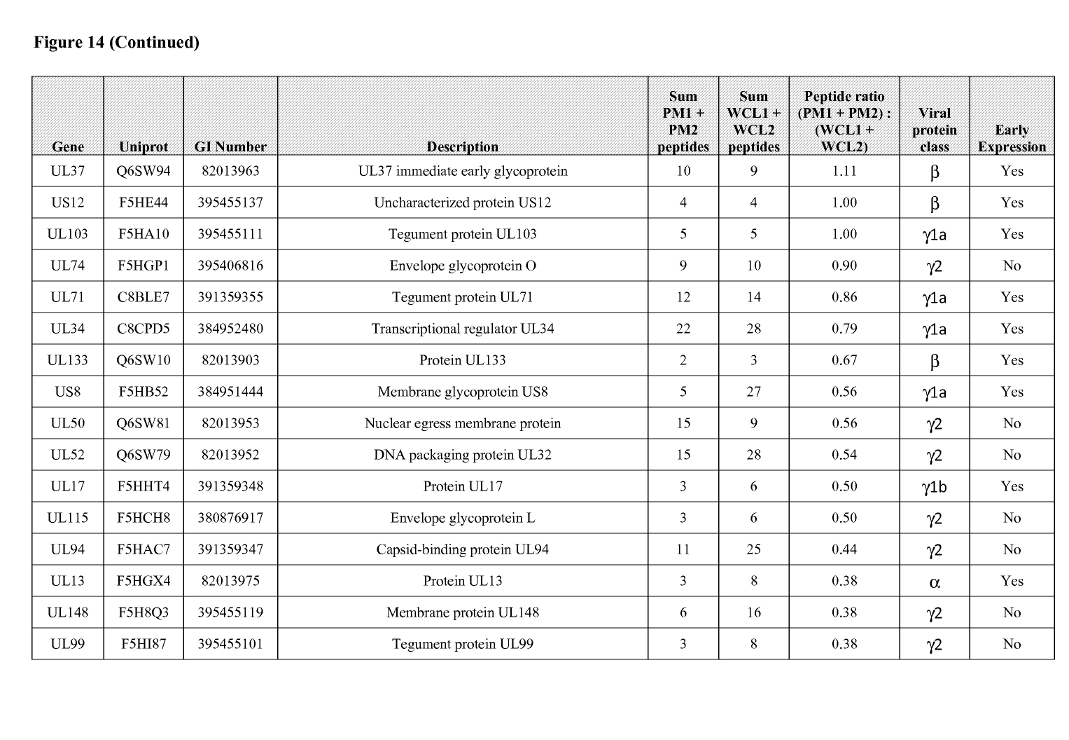

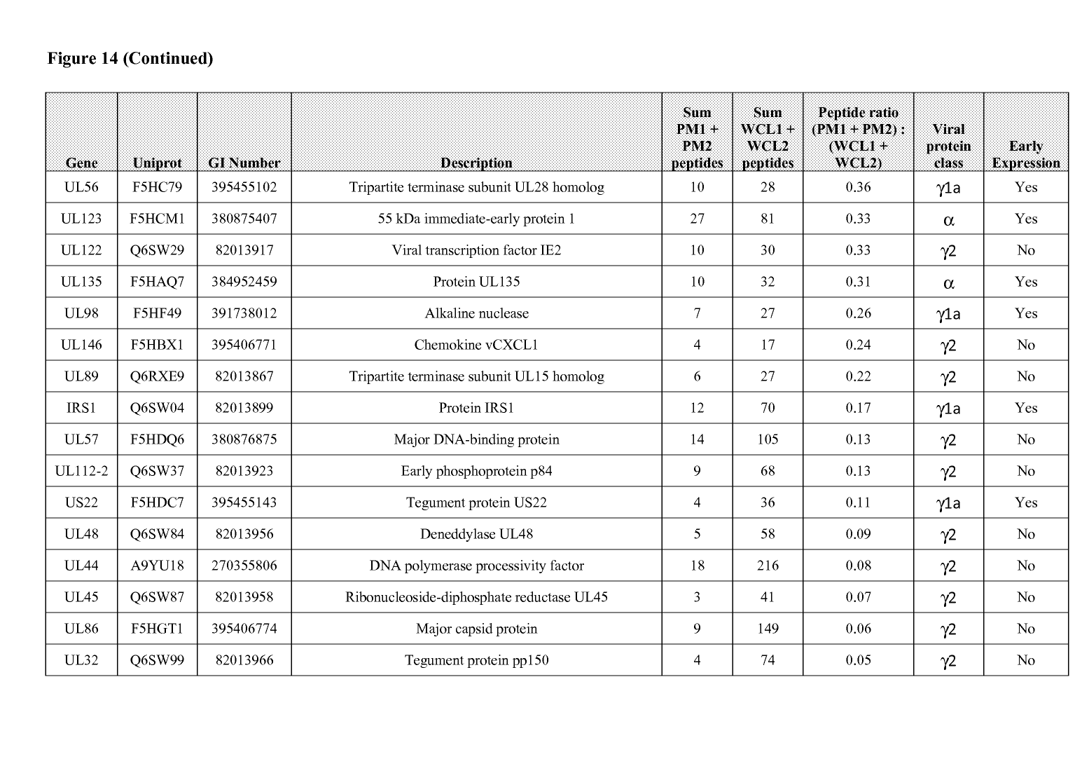

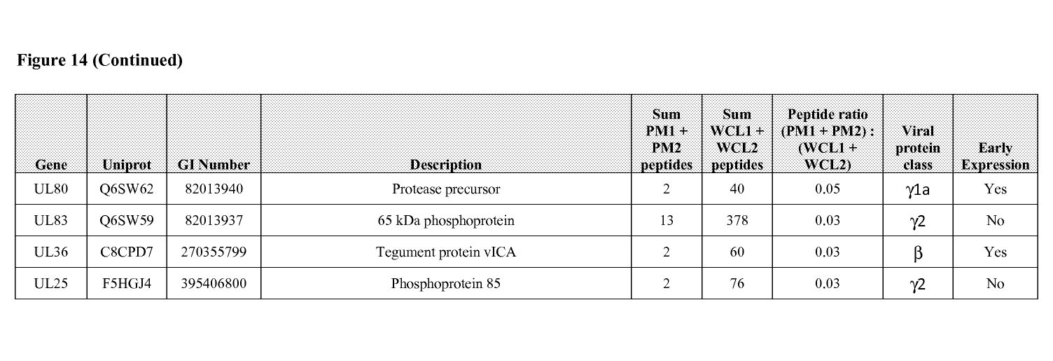

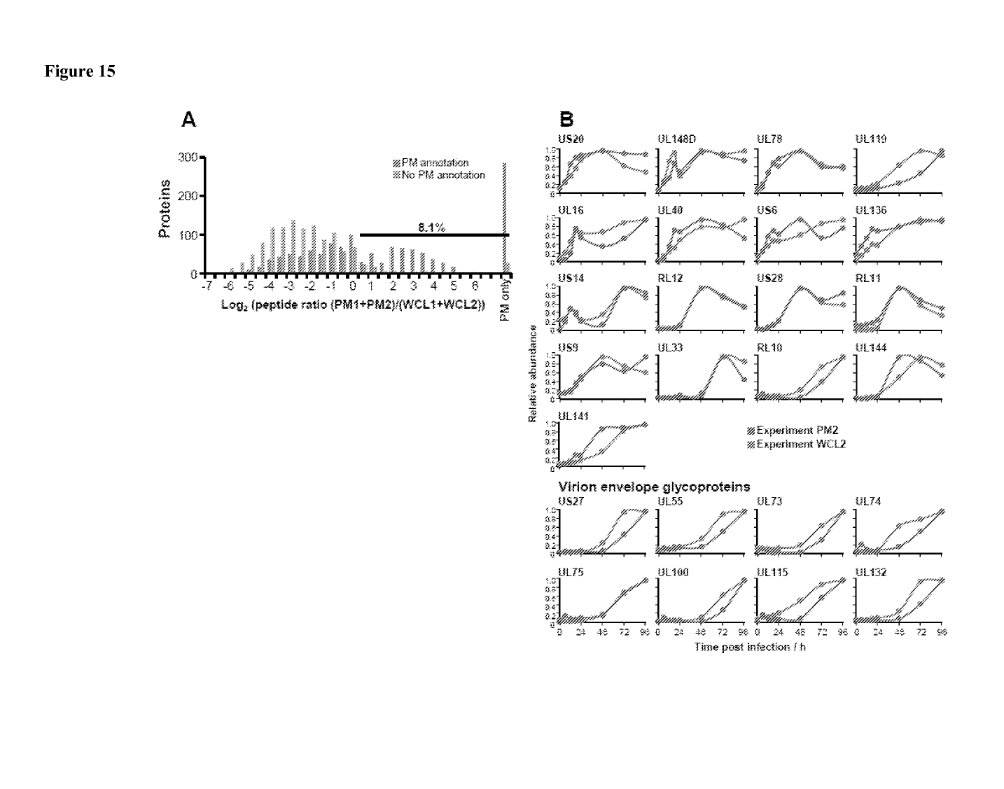

Viral proteins identified herein as present at the surface of infected cells are therapeutic targets. The majority of studies that have examined cell surface location of HCMV proteins have employed transduction of single viral genes, as opposed to productive infection. Only 6 HCMV proteins have been demonstrated at the PM of infected fibroblasts, all appearing late in infection, results that we confirmed (FIG. 3). A total of 67 viral proteins were detected in experiments PM1 and PM2. Subcellular localization of these proteins is poorly annotated, making it difficult to determine which may be non-PM contaminants, for example abundant viral tegument and nuclear proteins. A filtering strategy was used to screen out such contaminants: for every human Gene Ontology (GO)-annotated protein quantified in experiment PM1 or PM2, the ratio of peptides (PM1+PM2)/(WCL1+WCL2) was calculated. More than 90% of proteins without a PM GO annotation had a ratio of <1.4 (FIG. 15A). Applying this filter, 29 high confidence viral PM proteins were defined, which included the majority of viral proteins previously identified at the surface of either infected or transduced cells, and excluded all proteins unlikely to be present at the cell surface based on their known function (FIG. 14).

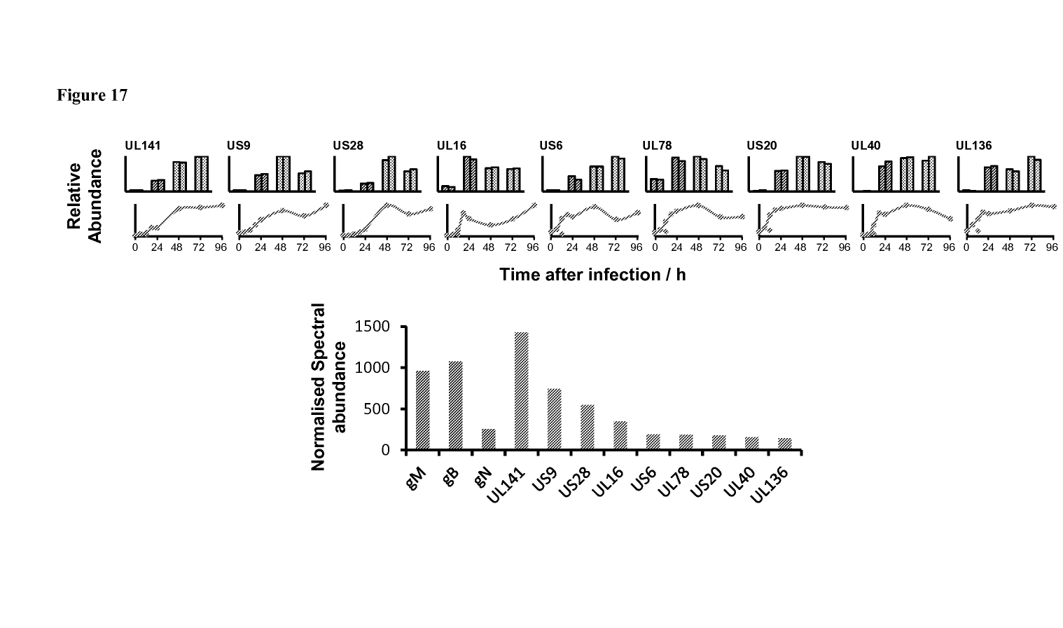

The high confidence viral PM proteins were assessed based on the following characteristics: (a) presence early in infection; (b) presence throughout the course of infection; and (c) sufficient abundance to distinguish infected from uninfected cells. Among the high confidence viral PM proteins, UL141, US9, US28, UL16, US6, UL78, US20, UL40 and UL136 best fit this criteria (FIG. 17).

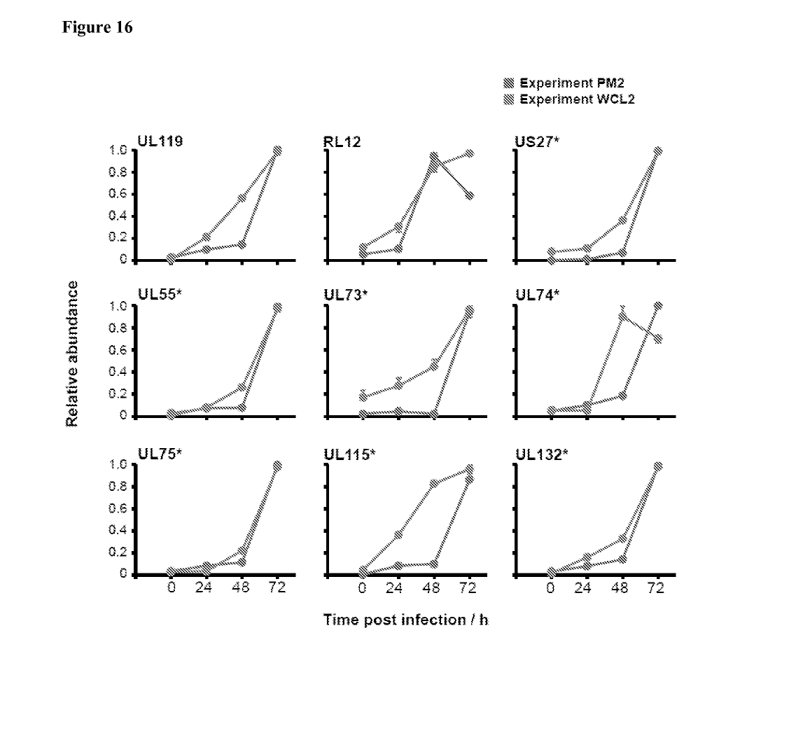

In general, a striking correlation between the PM2 and WCL2 temporal profiles of all 29 high confidence proteins was observed. For the subset of known virion envelope glycoproteins, protein appearance at the PM was significantly delayed compared to the WCL, confirmed by analysis of the same proteins from experiments PM1 and WCL1 (FIGS. 15B, 16). PM appearance of UL119 and RL10 was also delayed (FIG. 15B).

### Example 5

### HCMV Seropositive Serum Induces Antibody-Dependent Cytotoxicity

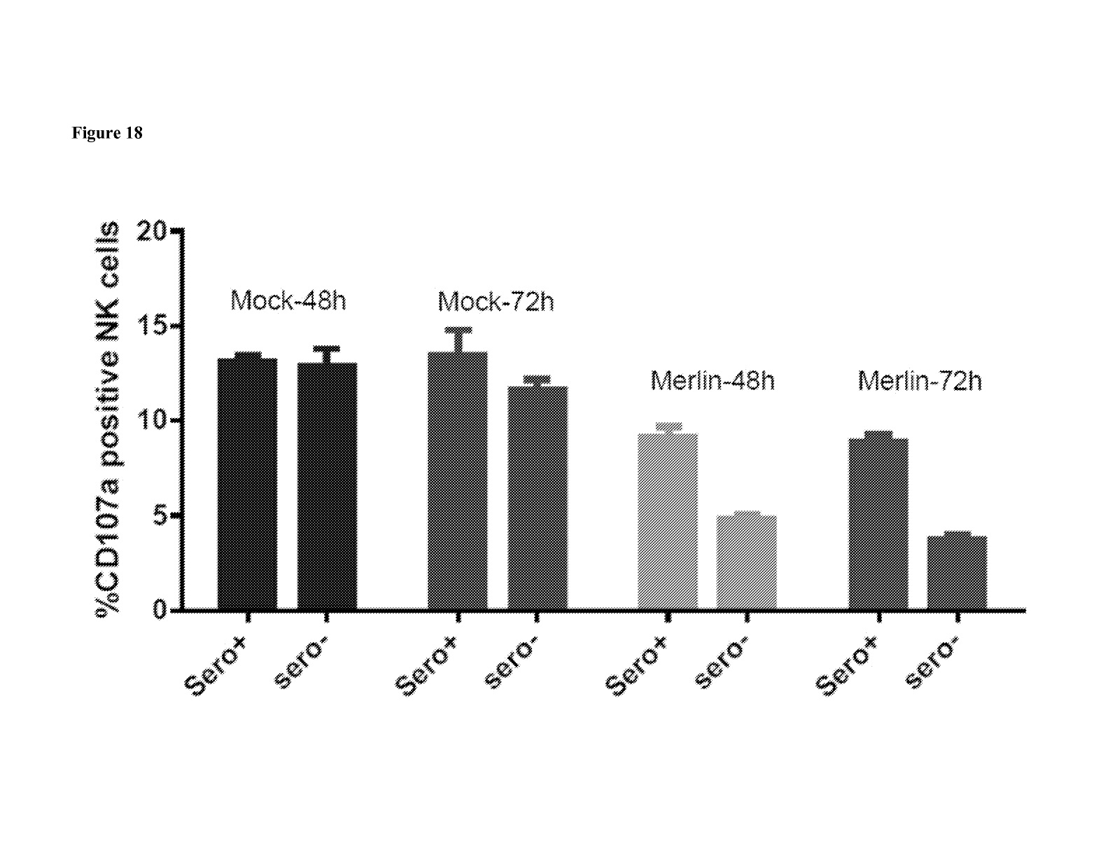

It was investigated whether serum from HCMV seropositive individuals induced antibody-dependent cytotoxicity (antibody-mediated lysis of virally-infected targets by NK cells). Fibroblasts were infected with HCMV strain Merlin. After 48 or 72 hours, NK cells and serum from HCMV seropositive or seronegative donors was added to the infected fibroblasts and the level of NK degranulation assessed in a CD107a assay. As seen in FIG. 18, NK cells showed approximately double the response to infected cells in the presence of seropositive serum, compared to seronegative serum, at both 48 and 72 hours post-infection. NK cells showed equal responses to Mock infected cells in the presence of both serums. This data indicates that the addition of serum from HCMV seropositive individuals (but not serum from seronegative individuals) induces antibody-dependent cellular cytotoxicity, supporting the use of therapeutic antibodies for the treatment of HCMV infection.

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

Those skilled in the art will recognize, or be able to ascertain using no more than routine experimentation, many equivalents to the specific embodiments of the invention described herein. Such equivalents are intended to be encompassed by the following claims.

1. A method of treating HCMV in a subject comprising administering to the subject an agent that specifically binds to a protein encoded by a gene selected from the genes listed in Table 1.

2. The method of claim 1, wherein the protein is encoded by a gene selected from the genes listed in Table 2.

3. The method of claim 1, wherein the agent is an antibody.

4. The method of claim 3, wherein the antibody is monoclonal.

5. The method of claim 3, wherein the antibody is chimeric, humanized or fully human.

6. The method of claim 3, wherein the antibody is selected from the group consisting of:
a full length immunoglobulin molecule;
an scFv;
a Fab fragment;
an Fab′ fragment;
an F(ab′)2;
an Fv;
a NANOBODY®; and
a disulfide linked Fv.

7. The method of claim 3, wherein the antibody binds to the protein with a dissociation constant of no greater than about 10⁻⁷ M.

8. The method of claim 3, wherein the antibody binds to an extracellular epitope of the protein.

9. The method of claim 8, wherein the epitope is selected from the epitopes listed in Table 5.

10. The method of claim 3, wherein the antibody is linked to a cytotoxic agent.

11. The method of claim 10, wherein the cytotoxic agent is selected from the group consisting of MMAE, DM-1, maytansinoids, doxorubicin derivatives, auristatins, calcheamicin, CC-1065, duocarmycins and anthracyclines.

12. The method of claim 3, wherein the antibody is linked to an antiviral agent.

13. The method of claim 12, wherein the antiviral agent is ganciclovir, valganciclovir, foscarnet, cidofovir, acyclovir, formivirsen, maribavir, BAY 38-4766 or GW275175X.

14. An antibody that specifically binds to an extracellular epitope of a protein encoded by a gene selected from the genes listed in Table 1.

15. The antibody of claim 14, wherein the protein is encoded by a gene selected from the genes listed in Table 2.

16. The antibody of claim 14, wherein the epitope is selected from the epitopes listed in Table 5.

17. The antibody of claim 14, wherein the antibody is monoclonal.

18. The antibody of claim 14, wherein the antibody is chimeric, humanized or fully human.

19. The antibody of claim 14, wherein the antibody is selected from the group consisting of:
a full length immunoglobulin molecule;
an scFv;
a Fab fragment;
an Fab′ fragment;
an F(ab′)2;
an Fv;
a NANOBODY®; and
a disulfide linked Fv.

20. The antibody of claim 14, wherein the antibody binds to the target protein with a dissociation constant of no greater than about 10⁻⁷ M.

21. The antibody of claim 14, wherein the antibody is linked to a cytotoxic agent.

22. The antibody of claim 21, wherein the cytotoxic agent is selected from the group consisting of MMAE, DM-1, maytansinoids, doxorubicin derivatives, auristatins, calcheamicin, CC-1065, duocarmycins and anthracyclines.

23. The antibody of 14, wherein the antibody is linked to an antiviral agent.

24. The antibody of claim 23, wherein the antiviral agent is ganciclovir, valganciclovir, foscarnet, cidofovir, acyclovir, formivirsen, maribavir, BAY 38-4766 or GW275175X.

25. A method of treating HCMV in a subject comprising administering to the subject an agent that specifically binds to a protein encoded by a group consisting of the genes listed in Table 3.

26. The method of claim 25, wherein the protein is encoded by a gene selected from the group consisting of CHST11, KCNK1, SPINT1, CDH1, CEACAM1, EPCAM, TNFRSF1B, ERBB3, CNTFR, PCDH1, BST2, SDK2, RALGPS2, SLCO4A1, MEGF10, SEMA4D, PCDH1, SPINT1 and TTC17.

27. The method of claim 25, wherein the agent is an antibody.

28. The method of claim 27, wherein the antibody is monoclonal.

29. The method of claim 27, wherein the antibody is chimeric, humanized or fully human.

30. The method of claim 27, wherein the antibody is selected from the group consisting of:
a full length immunoglobulin molecule;
an scFv;
a Fab fragment;
an Fab′ fragment;
an F(ab′)2;
an Fv;
a NANOBODY®; and
a disulfide linked Fv.

31. The method of claim 27, wherein the antibody binds to the protein with a dissociation constant of no greater than about 10⁻⁷ M.

32. The method of claim 27, wherein the antibody binds to an extracellular epitope of the protein.

33. The method of claim 27, wherein the antibody is linked to a cytotoxic agent.

34. The method of claim 33, wherein the cytotoxic agent is selected from the group consisting of MMAE, DM-1, maytansinoids, doxorubicin derivatives, auristatins, calcheamicin, CC-1065, duocarmycins and anthracyclines.

35. The method of claim 27, wherein the antibody is linked to an antiviral agent.

36. The method of claim 35, wherein the antiviral agent is ganciclovir, valganciclovir, foscarnet, cidofovir, acyclovir, formivirsen, maribavir, BAY 38-4766 or GW275175X.

37. An antibody that specifically binds to an extracellular epitope of a protein encoded by a group consisting of the genes listed in Table 3.

38. The antibody of claim 37, wherein the protein is encoded by a gene selected from the group consisting of CHST11, KCNK1, SPINT1, CDH1, CEACAM1, EPCAM, TNFRSF1B, ERBB3, CNTFR, PCDH1, BST2, SDK2, RALGPS2, SLCO4A1, MEGF10, SEMA4D, PCDH1, SPINT1 and TTC17.

39. The antibody of claim 37, wherein the antibody is monoclonal.

40. The antibody of claim 39, wherein the antibody is chimeric, humanized or fully human.

41. The antibody of claim 39, wherein the antibody is selected from the group consisting of:
a full length immunoglobulin molecule;
an scFv;
a Fab fragment;
an Fab′ fragment;
an F(ab′)2;
an Fv;
a NANOBODY®; and
a disulfide linked Fv.

42. The antibody of claim 39, wherein the antibody binds to the target protein with a dissociation constant of no greater than about 10⁻⁷ M.

43. The antibody of claim 39, wherein the antibody is linked to a cytotoxic agent.

44. The antibody of claim 43, wherein the cytotoxic agent is selected from the group consisting of MMAE, DM-1, maytansinoids, doxorubicin derivatives, auristatins, calcheamicin, CC-1065, duocarmycins and anthracyclines.

45. The antibody of claim 39, wherein the antibody is linked to an antiviral agent.

46. The antibody of claim 45, wherein the antiviral agent is ganciclovir, valganciclovir, foscarnet, cidofovir, acyclovir, formivirsen, maribavir, BAY 38-4766 or GW275175X.

47. A method of treating HCMV in a subject comprising administering to the subject a cytotoxic agent to which a protein encoded by ABCC3, SLC38A4 or SLC2A10 provides cellular resistance.

48. The method of claim 47, wherein the protein is encoded by ABCC3.

49. The method of claim 48, wherein the cytotoxic agent is Etoposide.

50. The method of claim 47, wherein the protein is encoded by SLC38A4.

51. The method of claim 47, wherein the protein is encoded by SLC2A10.
